(19) United States
(12) Patent Application Publication
(10) Pub. No.: US 2023/0326548 A1
(43) Pub. Date: Oct. 12, 2023

(54) UNSUPERVISED DISCOVERY OF TUMOR MICROENVIRONMENTAL COMMUNITIES

(71) Applicant: Washington University, St. Louis, MO (US)

(72) Inventors: Aadel Chaudhuri, St. Louis, MO (US); Erik Storrs, St. Louis, MO (US)

(73) Assignee: Washington University, St. Louis, MO (US)

(21) Appl. No.: 18/298,355

(22) Filed: Apr. 10, 2023

Related U.S. Application Data
(60) Provisional application No. 63/329,266, filed on Apr. 8, 2022.

(51) Int. Cl. G16B 20/00; G16B 40/30; G16H 20/40

(52) U.S. Cl. CPC G16B 20/00; G16B 40/30; G16H 20/40

(57) ABSTRACT

Systems and methods for the selection of a treatment for pancreatic adenocarcinoma (PDAC) in a patient in need based on single-cell RNA sequencing data obtained from a tumor biopsy sample obtained prior to treatment are disclosed. Also disclosed are systems and methods for predicting a clinical outcome of a pancreatic adenocarcinoma (PDAC) patient based on the single-cell RNA sequencing data.

### CROSS-REFERENCE TO RELATED APPLICATIONS

This application claims priority from U.S. Provisional Application Ser. No. 63/329,266 filed on Apr. 8, 2022, which is incorporated herein by reference in its entirety.

### STATEMENT REGARDING FEDERALLY SPONSORED RESEARCH OR DEVELOPMENT

This invention was made with government support under CA238711 awarded by the National Institutes of Health (NIH). The government has certain rights in the invention.

### MATERIAL INCORPORATED-BY-REFERENCE

Not applicable.

### FIELD OF THE DISCLOSURE

The present disclosure generally relates to systems and methods for the selection of a treatment for pancreatic adenocarcinoma (PDAC) in a patient in need based on single-cell RNA sequencing data obtained from a tumor biopsy sample obtained prior to treatment. The present disclosure further relates to systems and methods for predicting a clinical outcome of a pancreatic adenocarcinoma (PDAC) patient based on single-cell RNA sequencing data.

### BACKGROUND OF THE DISCLOSURE

Pancreatic ductal adenocarcinoma (PDAC) is the third leading cause of cancer death in the United States with a 5-year survival rate of 10.8%. PDAC has remained largely refractory to available therapeutics, with a hallmark of heterogenous chemotherapeutic responses in subsets of patients. Over the past decade, bulk tumor sequencing has enabled annotation of the genomic landscape in pancreatic ductal adenocarcinoma (PDAC). This has led to several classification systems for PDAC. The general consensus consistently demonstrates the existence of two major subtypes of PDAC: the classical or pancreatic progenitor subtype associated with a relatively better prognosis (characterized by differentiated ductal markers like PDX1) and the basal-like, squamous, or quasi-mesenchymal subtype associated with a poorer prognosis (characterized by the expression of basal-like markers like cytokeratin 81 (KRT81)). While these insights have allowed for the elucidation of unique transcriptional networks and therapy resistance, they have yet to allow for the development of effective clinical interventions.

Underlying this, in part, is the fact that these subtyping techniques rely on bulk sequencing data, creating blind spots in individual cell states and features of individual cells within a single tumor sample. This issue is especially pronounced in PDAC, where only 20% of cells are tumor cells, and thus the ability to fully decipher all cellular variants is limited when using traditional next-generation sequencing (NGS) methodologies. Underscoring the importance of granular analysis, recent experiments have demonstrated the complex interplay of subtypes of PDAC tumor cells with the associated stroma and the inflammasome. Advances in single-cell RNA sequencing (scRNA-seq) have provided the added ability to describe individual cell profiles and query individual cell states. These insights enable a more in-depth analysis of the tumor microenvironment (TME) and tumor heterogeneity with unparalleled granularity. Indeed, several scRNAseq efforts have demonstrated that PDAC tumors are a heterogeneous and spatially diverse admixture of “basal-like” and “classical” cells with a potential for plasticity between transcriptomic states with unknown prognostic implications. The tumor microenvironment drives transcriptional phenotypes and their plasticity in metastatic pancreatic cancer. Spatial drivers and pre-cancer populations collaborate with the microenvironment in untreated and chemo-resistant pancreatic cancer. Elucidation of tumor-stromal heterogeneity and the ligand-receptor interactome by single-cell transcriptomics in real-world pancreatic cancer biopsies. The majority of these efforts have focused on the utilization of surgical resection specimens or biopsies of patients with liver metastases or potentially from archival material. Single-nucleus and spatial transcriptomics of archival pancreatic cancer reveal multi-compartment reprogramming after neoadjuvant treatment rather than focusing on time-of-diagnosis endoscopic ultrasound (EUS) needle biopsies in resectable or locally advanced patients. Integrating these technologies with standard-of-care tissue acquisition at the time of diagnosis may allow these granular insights to serve as potential biomarkers for personalized therapeutics.

Other objects and features will be in part apparent and in part pointed out hereinafter.

### SUMMARY OF THE DISCLOSURE

Among the various aspects of the present disclosure is the provision of a method of unsupervised discovery of tumor microenvironmental communities.

Briefly, therefore, the present disclosure is directed to methods of selecting a cancer treatment by characterizing a patient's tumor microenvironment.

In various aspects. a computer-implemented method of selecting a treatment for pancreatic adenocarcinoma (PDAC) in a patient in need is disclosed, that includes receiving, at a computing device, a single-cell RNA sequencing (scRNA-seq) dataset comprising at least one RNA expression signature and associated cell state and a bulk RNA sequencing sample derived from a tumor sample obtained from the patient; transforming, using the computing device, the bulk RNA sequencing sample into a cell state fraction dataset comprising a plurality of cell states and associated proportion of the expression of the scRNA-seq dataset attributable to each cell state; assigning, using the computing device, one tumor microenvironment (TME) cell state from a TME dataset to the patient based on the cell fraction dataset; and selecting a treatment for the patient based on the assigned TME cell state. In some aspects, each TME cell state of the TME dataset comprises a unique distribution of cell fractions among the plurality of cell state categories. In some aspects, each TME cell state further comprises a predicted clinical outcome associated with each TME cell state. In some aspects, the method further includes producing a TME dataset by receiving, at the computing site, a plurality of calibration single-cell RNA sequencing (scRNA-seq) datasets and associated clinical outcome measurements obtained from at least one PDAC patient population; transforming, using the computing device, each calibration scRNA-seq dataset into a calibration cell fraction dataset comprising a plurality of cell states and associated proportion of the expression of the scRNA-seq dataset attributable to each cell state; assigning, using the computing device, the plurality of calibration cell fraction datasets to a TME cell state of the TME dataset, wherein the TME cell state comprises a cell fraction distribution shared by all calibration cell fraction datasets assigned to the TME cell state; and associating, using the computing device, a clinical outcome to the TME cell state, the clinical outcome comprising the shared clinical outcome associated with all calibration cell fraction datasets assigned to the TME cell state. In some aspects, selecting a treatment for the patient based on the assigned TME cell state includes selecting an immune checkpoint blockade treatment if the assigned TME cell state is indicative of an immune-enriched tumors; and selecting a treatment comprising administration of an active compound targeting a molecular pathway specific to an immature malignant cell state if the assigned TME cell state is indicative of a genomically less differentiated tumor.

The present teachings include methods for selecting a treatment for pancreatic adenocarcinoma (PDAC) in a patient in need. In one aspect, the methods can be computer-implemented. In another aspect, the method can include receiving, at a computing device, a single-cell RNA sequencing (scRNA-seq) dataset obtained from the patient. In another aspect, the method can include transforming, using the computing device, the scRNA-seq dataset into a cell fraction dataset that includes a plurality of cell states and associated proportion of the expression of the scRNA-seq dataset attributable to each cell state. In another aspect, the method can include assigning, using the computing device, one tumor microenvironment (TME) cell state from a TME dataset to the patient based on the cell fraction dataset. In yet another aspect, the method can include selecting a treatment for the patient based on the assigned TME cell state. In accordance with another aspect, each TME cell state of the TME dataset can be a unique distribution of cell fractions among the plurality of cell state categories. In another aspect, each TME cell state can further include a predetermined clinical outcome associated with each TME cell state. In another aspect of the present disclosure, the method can include producing a TME dataset. In one aspect, the method of producing a TME dataset can include receiving, at the computing site, a plurality of calibration single-cell RNA sequencing (scRNA-seq) datasets and associated clinical outcome measurements obtained from at least one PDAC patient population. In another aspect, the method can include transforming, using the computing device, each calibration scRNA-seq dataset into a calibration cell fraction dataset that includes a plurality of cell states and associated proportion of the expression of the scRNA-seq dataset attributable to each cell state. In one more aspect, the method can include assigning, using the computing device, the plurality of calibration cell fraction datasets to a TME cell state of the TME dataset, wherein the TME cell state can be a cell fraction distribution shared by all calibration cell fraction datasets assigned to the TME cell state. In yet another aspect, the method can include associating, using the computing device, a clinical outcome to the TME cell state. In one aspect, the clinical outcome can be the shared clinical outcome associated with all calibration cell fraction datasets assigned to the TME cell state.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a schematic of the RNAseq methods described in the present disclosure. Single-cell RNA-seq (scRNA-seq) was performed on treatment-naïve pancreatic adenocarcinoma (PDAC) tumor samples acquired by EUS-FNB (n=22) or surgical resection (n=6). These were integrated with samples from three publicly available PDAC scRNA-seq datasets resulting in a combined dataset of ˜150 k cells from 77 independent patient samples. The resulting data were used to identify PDAC cell states, including malignant and immune subtypes based on gene sets and known expression markers from published studies. With single-cell annotations in hand, the digital deconvolution tool CIBERSORTx was used to infer cell state fractions across 391 independent PDAC tumor samples across four publicly available bulk RNA-seq datasets. Unsupervised clustering was then utilized, and EcoTyper was orthogonally applied, to identify patterns of cell states in PDAC with predictive and prognostic significance.

FIG. 6A is a UMAP decomposition of expression profiles from ˜150 k cells representing 14 cell types present in an in-house and four publicly available scRNA-seq datasets (pancreatic ductal adenocarcinoma).

FIG. 6B is a UMAP decomposition of 5 malignant subtypes from ˜150 k cells representing 14 cell types present in an in-house and four publicly available scRNA-seq datasets (pancreatic ductal adenocarcinoma).

FIG. 6C is a UMAP decomposition of 5 immune cell states from ˜150 k cells representing 14 cell types present in an in-house and four publicly available scRNA-seq datasets (pancreatic ductal adenocarcinoma).

FIG. 6D is a heatmap comparison of the scRNA-seq-derived malignant subtype expression profiles in the present disclosure to published bulk RNA-seq subtypes.

FIG. 6E is a heatmap comparison of scRNA-seq immune cell states to known expression markers.

FIG. 6F is a CytoTRACE differentiation continuum across the 5 malignant single-cell subtypes.

FIG. 6G is a graph of CytoTRACE developmental scores that differ between the two subtypes of Classical tumor cells identified in the scRNA-seq data.

FIG. 7A is a schema for the generation of an expression signature matrix from scRNA-seq-derived cellular subtypes and the deconvolution of bulk RNA-seq data with CIBERSORTx.

FIG. 7B is a UMAP decomposition of deconvolved bulk RNA-seq samples colored by tumor microenvironmental community and dataset.

FIG. 7C is a plot showing cell state fractions in each TME community. Red boxes represent significantly enriched malignant, immune, or stromal cell fractions (P<0.05).

FIG. 8A is a schema showing how expression profiles from in-house scRNA-seq data were used to discover cell state fractions and gene expression profiles for samples were reconstructed in four publicly available bulk RNA-seq datasets.

FIG. 8B is a heatmap showing ecotype cell state abundances for each bulk RNA-seq sample across six discovered ecotypes.

FIG. 8C is a plot showing the overlap between ecotypes and TME communities identified within bulk RNA-seq samples.

FIG. 9A is a heatmap of deconvolved cell state fractional abundances for each TME community in bulk RNA-seq data, correlated with EcoTyper-discovered malignant cell states. −log 2 p-values for associations with overall survival are also shown.

FIG. 9B is a plot of Kaplan-Meier curves of EcoTyper-discovered CD8 T cell states.

FIG. 9C is a plot of gene set enrichment analysis of EcoTyper-discovered CD8 T cell state with the highest overall survival that shows enrichment for immune cell activating pathways.

FIG. 9D is a heatmap of the correlation between deconvolved bulk RNA-seq malignant subtype cell fractions and EcoTyper-derived CD8 T cell states that shows enrichment of S02/S03 in basal and mixed communities and absence of the immune-activating S01 cell state.

FIG. 9E is a graph of the correlation of the top 20 genes associated with each EcoTyper-discovered malignant cell state with differentiation level inferred by CytoTRACE.

FIG. 9F is a plot of Kaplan-Meier curves of EcoTyper-discovered malignant cell states.

FIG. 10A is a plot showing the overlap of Ecotype E1 with TME communities.

FIG. 10B is a plot of Kaplan-Meier curves of EcoTyper-discovered ecotypes showing increased overall survival in patients harboring immune- and stromal-high PDAC tumors.

FIG. 10C is a plot of Kaplan-Meier curves of TME communities showing increased overall survival in patients harboring immune- and stromal-high PDAC tumors.

FIG. 11A is a UMAP decomposed expression profile overlaid with dataset membership for all cell types.

FIG. 11B is a UMAP decomposed expression profile overlaid with dataset membership for malignant cells.

FIG. 11C is a UMAP decomposed expression profile overlaid with dataset membership for immune cells.

FIG. 12A is a UMAP decomposed expression profile overlaid with subtype classification.

FIG. 12B is a UMAP decomposed expression profile overlaid with Moffit subtype scores.

FIG. 12C is a UMAP decomposed expression profile overlaid with Collison subtype scores.

FIG. 12D is a UMAP decomposed expression profile overlaid with Bailey subtype scores.

FIG. 13A is a plot of cell state fractions for pseudo-bulk deconvolved cell state fractions and scRNA-seq manually annotated cell fractions for 22 EUS/FNB samples.

FIG. 13B is a plot of the correlation of pseudo-bulk deconvolved and manually annotated cell state fractions for each deconvolved cell state.

FIG. 14A is a plot of deconvolved EUS/FNB pseudo-bulk cell state fractions for the Malignant—Basal cell state against the CD4/CD8 T cell—exhausted cell state showing a positive association between Malignant—Basal and exhausted CD4/CD8 T cell abundance, while a negative association is present between levels of activated EcoTyper activated S01 CD8 T cells and exhausted CD4/CD8 T cells

FIG. 14B is a plot of deconvolved EUS/FNB pseudo-bulk cell state fractions for the EcoTyper activated S01 CD8 T cell state against the CD4/CD8 T cell—exhausted cell state showing a positive association between Malignant—Basal and exhausted CD4/CD8 T cell abundance, while a negative association is present between levels of activated EcoTyper activated S01 CD8 T cells and exhausted CD4/CD8 T cells.

FIG. 14C is a heatmap of the Spearman correlation between abundances for CIBERSORTx malignant cell states and Ecotyper-discovered malignant cell states, along with the correlation between CIBERSORTx malignant cell states and overall survival for the EUS/FNB samples.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, methods of diagnosing, selecting a treatment, and/or predicting a clinical outcome of a patient with pancreatic ductal adenocarcinoma (PDAC) based on single-cell RNA sequencing of a PDAC tissue biopsy sample are disclosed.

The disclosed method includes obtaining, producing, or receiving a single-cell RNA sequencing (scRNA-seq) dataset obtained from the patient that includes an RNA expression profile indicative of the relative proportions of RNA sequences detected within the sample. The disclosed method further includes transforming the scRNA-seq dataset into a cell fraction dataset comprising a plurality of cell states and the associated proportion of the expression of the scRNA-seq dataset attributable to each cell state. In various aspects, the cell fraction dataset may be obtained by assigning each scRNA sequence of the dataset to a cell state and deconvoluting the annotated scRNA-seq into the cell fraction dataset. The method further includes assigning one tumor microenvironment (TME) cell state from a TME dataset to the patient based on the cell fraction dataset.

In various aspects, TME cell states of the TME dataset are produced by an analysis of the calibration dataset comprising a plurality of calibration single-cell RNA sequencing (scRNA-seq) datasets and associated clinical outcomes from a patient population as described herein. In various other aspects, each TME cell state of the TME dataset includes a unique distribution of cell fractions among the plurality of cell state categories and is associated with a predetermined clinical outcome.

In various other aspects, methods for the computer-aided identification of microenvironmental communities associated with enhanced survival of cancer including, but not limited to pancreatic ductal adenocarcinoma (PDAC) are disclosed herein.

As described herein, scRNA-sequencing of PDAC tumor tissues from standard endoscopic ultrasound-guided fine needle biopsy (EUS-FNB) specimens at the time of diagnosis and from surgical samples from tumor resections were performed. The scRNA-sequencing results were integrated with samples from three additional publicly available RNA-seq studies. Utilizing this approach, tumor microenvironment (TME) cell states and previously described major molecular subtypes were identified in the single-cell data. The CytoTRACE algorithm, an innovative method for inferring developmental cell states, including potential cancer stem cells, was then applied to reveal a developmental dichotomy within classical tumor cells.

To clinically translate the cell states determined as described above from the single-cell expression data to patient outcomes, CIBERSORTx, a method for inferring cell state fractions in bulk RNA-seq and microarray samples, was used to infer cell type fractions in four publicly available datasets. Unsupervised clustering techniques and EcoTyper were used to identify “communities” and ecotypes associated with overall survival. Patterns of cell state abundance found in deconvolved bulk expression datasets were also evident in pseudo-bulked deconvolved EUS-FNB samples. The analysis systems and methods described herein revealed new predictive insights into PDAC starting from the time of diagnosis, paving the way toward personalized risk-adapted therapy using upfront genomic features analysis.

In various aspects, at least a portion of the disclosed methods may be implemented using various computing systems and devices as described below.

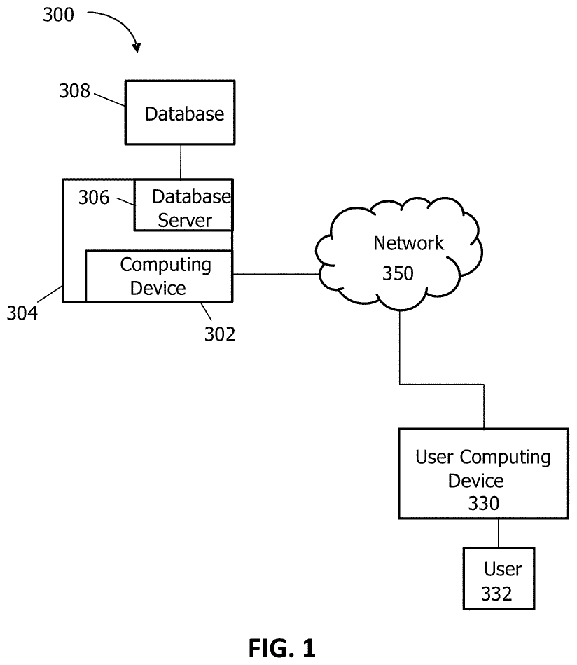

FIG. 1 depicts a simplified block diagram of a computing device for implementing the methods described herein. As illustrated in FIG. 1, the computing device 300 may be configured to implement at least a portion of the tasks associated with the disclosed method. The computer system 300 may include a computing device 302. In one aspect, the computing device 302 is part of a server system 304, which also includes a database server 306. The computing device 302 is in communication with database 308 through the database server 306. The computing device 302 is communicably coupled to a user-computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user-computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

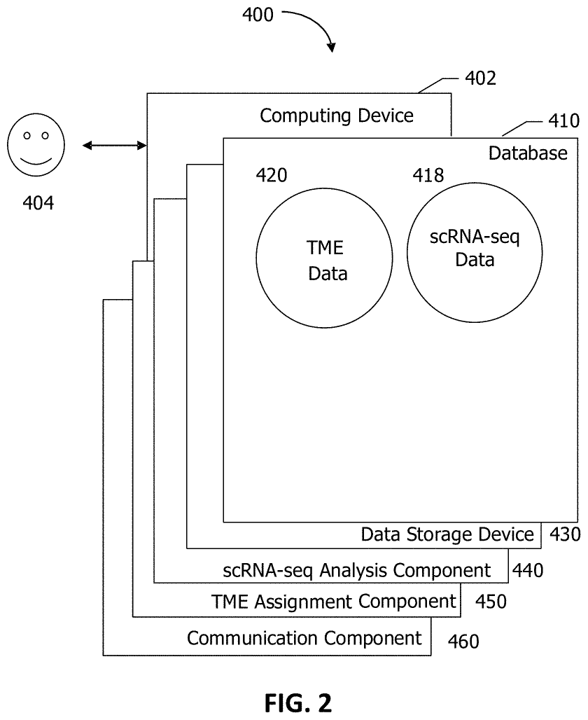

In other aspects, the computing device 302 is configured to perform a plurality of tasks associated with the disclosed method. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes scRNA-seq data 418 and TME assignment data 420. Non-limiting examples of suitable scRNA-seq data 418 include a patient scRNA-seq dataset, a calibration scRNA-seq dataset, and any other scRNA-seq dataset as needed to implement the disclosed method in any aspect. Non-limiting examples of suitable TME data 420 include the TME dataset and associated clinical outcomes, treatment recommendations, prognoses, and any other data needed to select a treatment and/or predict a clinical outcome based on a scRNA-seq dataset of a patient using the systems and methods disclosed herein.

Computing device 402 also includes a number of components that perform specific tasks. In the exemplary aspect, computing device 402 includes a data storage device 430, a scRNA-seq analysis component 440, a TME assignment component 450, and a communication component 460. Data storage device 430 is configured to store data received or generated by computing device 402, such as any of the data stored in database 410 or any outputs of processes implemented by any component of computing device 402. The scRNA-seq analysis component 440 is configured to analyze a patient's scRNA-seq dataset to obtain a cell fraction dataset using the systems and methods disclosed herein. The TME assignment component 450 is configured to assign the patient's cell fraction dataset to a TME cell state and to select a treatment or predict a clinical outcome for the patient based on the assigned TME cell state.

Communication component 460 is configured to enable communications between computing device 402 and other devices (e.g. user computing device 330 and sequencing system 310, shown in FIG. 1) over a network, such as network 350 (shown in FIG. 1), or a plurality of network connections using predefined network protocols such as TCP/IP (Transmission Control Protocol/Internet Protocol).

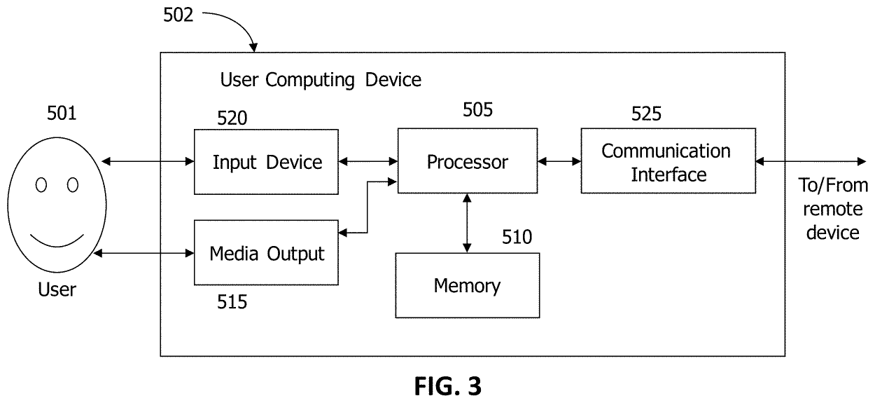

FIG. 3 depicts a configuration of a remote or user-computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

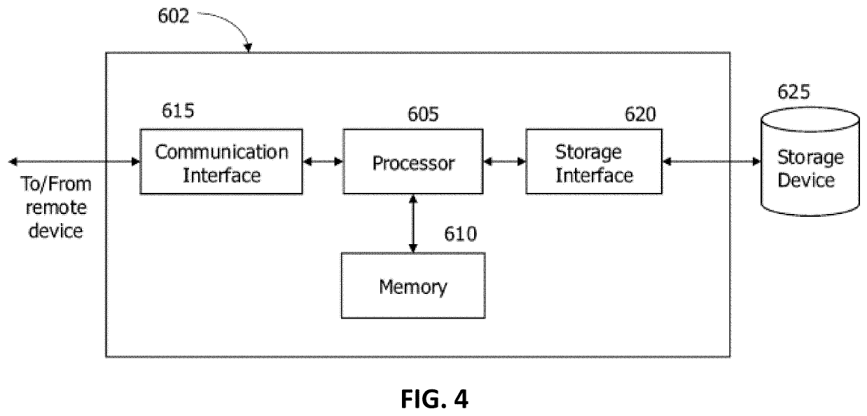

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated in server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may further include: sequencing data, sensor data, image data, video data, telematics data, authentication data, authorization data, security data, mobile device data, geolocation information, transaction data, personal identification data, financial data, usage data, weather pattern data, “big data” sets, and/or user preference data. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are examples only, and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—High-Dimensional Deconstruction of Pancreatic Ductal Adenocarcinoma Identifies Tumor Microenvironmental Communities Associated with Survival

Abstract

Pancreatic ductal adenocarcinoma (PDAC) is among the deadliest cancers worldwide. Bulk and single-cell technologies have recently been leveraged to better understand its genomic underpinnings. The PDAC tumor microenvironment (TME) has also been explored, revealing an immunosuppressive milieu. However, efforts to utilize TME features to facilitate more effective treatments have largely failed. Here, single-cell RNA sequencing (scRNA-seq) is performed on a cohort of treatment-naive PDAC biopsy samples (n=22) and surgical samples (n=6), integrated with 3 public datasets (n=49), resulting in 140,000 individual cells from 77 patients. Based on expression markers assessed by Seurat v 3 and differentiation status assessed by CytoTRACE, the resulting tumor cellular clusters are divided into 5 molecular subtypes: Basal, Mixed Basal/Classical, Less differentiated Classical, More differentiated Classical, and ADEX. These 5 tumor cell profiles are then queried, along with 15 scRNA-seq-derived tumor microenvironmental cellular profiles, in 391 bulk RNA-seq samples from 4 published datasets of localized PDAC with associated clinical metadata using CIBERSORTx. Through unsupervised clustering analysis of these 20 cell state fractions representing tumor, leukocyte, and stromal cells, 7 unique clustering patterns are identified representing combinations of tumor cellular and microenvironmental cell states present in PDAC tumors, termed communities, and these patterns are correlated with overall survival, tumor ecotypes, and tumor cellular differentiation status.

In the present Example, 7 distinct cellular communities were identified in bulk RNA sequencing data after CIBERSORTx deconvolution and unsupervised clustering. The community associated with the worst overall survival contained basal tumor cells, exhausted CD4 and CD8 T cells, and was enriched for fibroblasts. In contrast, the highest overall survival was associated with a community enriched for differentiated classical tumor cells, NK cells, and endothelial cells. The differentiation state of tumor cells (assessed by CytoTRACE) also correlated with survival in a dose-dependent fashion. The community structures identified with the unsupervised clustering approach were corroborated with ecotypes obtained using EcoTyper and a significant correlation was observed. A subset of PDAC samples was identified that were significantly enriched for activated CD8 T cells that achieved a 3-year overall survival rate of 40%, suggesting PDAC patients with improved prognoses and with potentially higher sensitivity to immunotherapy can be identified. Discovered tumor microenvironmental communities from high-dimensional analysis of PDAC RNA sequencing data reveal new connections between tumor microenvironmental composition and patient survival that could lead to better upfront risk stratification and more personalized clinical decision-making.

### Introduction

Here, scRNA-sequencing of PDAC from standard endoscopic ultrasound-guided fine needle biopsy (EUS-FNB) specimens is performed at the time of diagnosis and from surgical samples from tumor resections. These cases are then integrated with samples from three additional publicly available RNA-seq studies. Utilizing this approach, TME cell states and previously described major molecular subtypes are identified in the single-cell data. The CytoTRACE algorithm is then applied, an innovative method for inferring developmental cell states, including potential cancer stem cells, which reveals a developmental dichotomy within classical tumor cells. To clinically translate the cell states found in the single cell expression data to patient outcomes, CIBERSORTx, a method for inferring cell state fractions in bulk RNA-seq and microarray samples, is used to infer cell type fractions in four publicly available datasets. Unsupervised clustering techniques and EcoTyper are then used to identify “communities” and ecotypes found to be associated with overall survival. Finally, it is shown that patterns of cell state abundance found in deconvolved bulk expression datasets are also evident in pseudo-bulked deconvolved EUS-FNB samples. The innovative approach reveals new predictive insight into PDAC starting from the time of diagnosis, thus paving the way toward personalized risk-adapted therapy using upfront genomic features analysis.

### Methods

### PDAC Tumor Processing

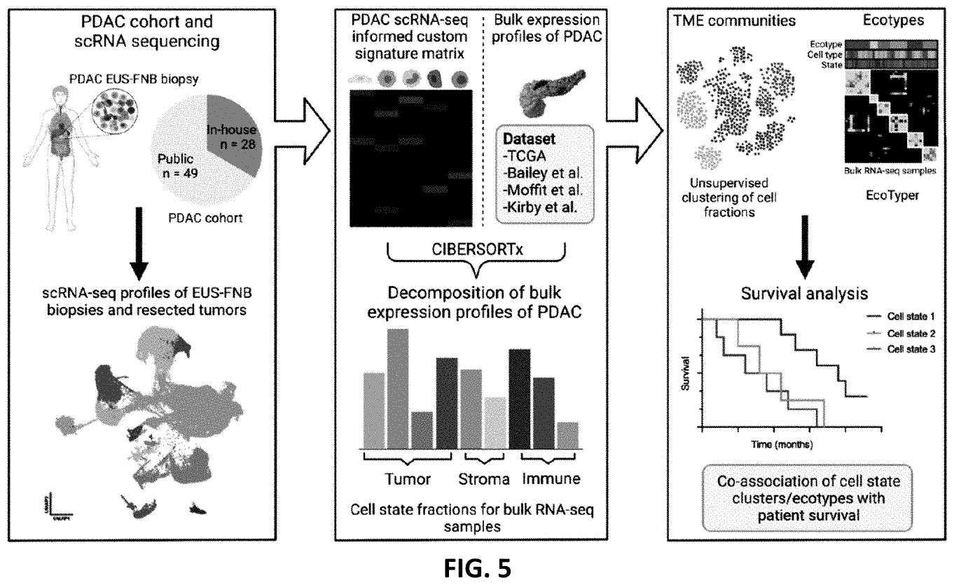

Endoscopic ultrasound was performed on patients with suspected solid pancreatic masses based on CT or MRI imaging (FIG. 5). The diagnosis of pancreatic adenocarcinoma was confirmed by a formal pathologic evaluation. After clinical diagnostic tissue acquisition was completed with 2-3 passes of a 22-gauge needle, an additional pass was obtained with a backfin “fine-needle biopsy” (FNB) needle. Tissue was carefully washed with cold PBS, collected in RPMI 1640 media (Gibco) on ice and dissociated into single-cell suspensions both mechanically and enzymatically. Resected surgical tumor tissue was also dissociated in a similar way to obtain single-cell suspensions. Subsequently, single-cell suspensions were prepared to a final concentration of ˜1,000 cells/μl, and sequencing libraries were prepared using the 10× Genomics Chromium Single Cell 5′ library platform. Complementary DNA libraries were then sequenced on an Illumina NovaSeq S4 flow cell with a target median depth of 50,000 reads/cell.

In-House scRNA-Seq Data Processing

Reads were aligned to the GRCh38 reference genome and gene expression counts were obtained using 10× Cell Ranger 3.0.2 with default parameters. FASTQ files were aligned to the GRCh38 reference genome with the STAR aligner. Cell-specific unique molecular identifiers (UMIs) were then used to generate gene expression matrices. Cells that expressed less than 200 total genes and genes that were expressed in fewer than 3 cells were filtered from the dataset. Additionally, cells with a feature count of over 7,500 or a mitochondrial DNA percentage of over 20% were also filtered from the dataset.

Integration of Public scRNA-Seq Datasets

Filtered In-house EUS-FNB and surgical samples were integrated with three publicly available datasets. These datasets include Peng et al. (n=24), Lin et al. (n=10), and Chan-Seng-Yue et al. (n=15). Expression counts were downloaded for these datasets and integrated using anchor transfer methodology implemented by the Seurat single-cell library (https://www.cell.com/cell/fulltext/S0092-8674(19)30559-8). Each dataset was first normalized with the SCTransform function. The 3,000 most variable features to use for anchor transfer with the function SelectIntegrationFeatures were identified and the dataset integration was performed using the functions FindIntegrationAnchors and IntegrateData.

### Cell State Identification

The first 30 principle components (determined via RunPCA) of the integrated dataset were used for nearest neighbor computation, UMAP dimensionality reduction, and clustering. The Louvain algorithm was used to cluster single-cell expression data via the FindClusters Seurat function. For initial clustering, a resolution of 0.75 was used. Clusters were identified and merged based on known cell type expression markers: CAF (BGN+, FAP+), T-cell (CD45+, CD3G+), DC (FCER1A+, CD74+, HLA-DRA+), Malignant (EPCAM+, KRT18+), Endothelial cell (PECAM1+), Erythrocyte (HBA1+), B cell (KIT+), Mast cell (CPA3+), Monocyte (FCER1A+, CD14+, LYZ+), Plasma cell (SDC1+), Acinar (PRSS1+, CDHS+), Stellate I and II (RGSS+). The T cell cluster was further clustered with a resolution of X. The resulting clusters were grouped into CD4 T cells (CD3G+, CD4+), CD8 T cells (CD3G+, CD8A+), CD4/CD8 T cells—exhausted (CD3G+, LAG3+, PDCD1+), NK (NKG7+, GNLY+), and T-reg (FOXP3+).

### Identification of PDAC Subtypes

Malignant cells were clustered with a resolution of 0.1. They were then labeled based on subtype markers previously described in the literature. 3 gene marker sets (Bailey, Moffitt, and Collisson) were used for cluster assignment. For each subtype in Bailey and Moffitt, 20 genes were used. From the Collisson gene set, all available genes were used. We then came up with a composite score for each subtype in each marker set by taking the mean expression of all genes in the marker set. The labels Basal, Mixed Basal/Classical, Classical I (low classical), Classical II (high classical), and ADEX were given based on enrichment for these marker gene sets.

### Bulk Expression Data Acquisition

TCGA PDAC clinical and bulk RNA-seq expression data were downloaded from the NCI GDC (https://portal.gdc.cancer.gov/); samples were restricted to those used from TCGA, Cancer Cell, 2017 (n=136). Bailey et al PDAC clinical and bulk RNA-seq expression data (n=87) were downloaded from the ICGC Data Portal (https://dcc.icgc.org/projects/PACA-AU). Moffit et al (n=123) microarray expression and clinical data were downloaded from the Gene Expression Omnibus under the accession number GSE71729 (https://www.ncbi.nlm.nih.gov/geo/query/acc.cgi?acc=GSE71729). Kirby et. al. was downloaded. Samples from all datasets where survival time was not present or was less than 1 month were not used in the analysis.

### Bulk Expression Deconvolution

Signature matrices were derived from the annotated single-cell expression data, which were input as raw counts. Bulk RNA-seq data, prior to CIBERSORTx input, were normalized to the total number of counts (CPM) for RNA-seq data (TCGA, Bailey, and Kirby), while microarray data (Moffit) was kept as downloaded.

CIBERSORTx was run in a containerized form via Docker with the following parameters—single_cell TRUE—rmbatchSmode TRUE.

### Unsupervised Community Detection and Analysis

After computing cell fractions with CIBERSORTx, a batch correction was applied to the samples from the different datasets to account for technical differences in the dataset. To this end, the cell fractions were integrated by projecting them via a PCA that was fitted to cell fractions from samples from Bailey et al. The resulting embeddings were then mapped with UMAP. Samples from Bailey et al. were clustered with the Leiden algorithm with a resolution of 0.8. The clusters were then transferred via a KNN algorithm. This functionality was implemented in the scanpy.tl.leiden and scanpy.tl.ingest functions.

Enrichment for cell fraction was computed via the Wilcoxon rank-sum test. Significance levels were adjusted via the Benjamini-Hochberg method. Functionality was implemented in the scanpy.tl.rank_genes_groups function.

### EcoTyper Ecotype Discovery

Ecotype discovery was performed with EcoTyper. Cell states and ecotypes were discovered on the TCGA bulk RNA-seq dataset according to steps in EcoTyper documentation Tutorial #4: De novo Discovery of Cell States and Ecotypes in Bulk Expression Data. Briefly, cell fraction estimation was performed with CIBERSORTx on scRNA-seq expression profiles from previously-identified cell states described herein. To allow EcoTyper to discover its own malignant cell states, all malignant cells were labeled as malignant, rather than their subtype-specific classification. Following cell fraction estimation, steps for EcoTyper cell state and ecotype discovery were performed. Steps in Tutorial #1: Recovery of Cell States and Ecotypes in User-Provided Bulk Data were then executed for Bailey, Moffit, and Kirby datasets.

Overlap of discovered ecotypes and communities was computed as the ratio of the number of samples present in ecotype and community/total number of samples in ecotype.

### Survival Analysis

Kaplan Meier curves and log rank p-values were computed with the Python lifelines package.

For survival analysis of Malignant cell fractions and EcoTyper discovered cell states, log rank p-values were computed.

Gene Set Enrichment Analysis (GSEA)

Pathway enrichment analysis for significantly associated genes with the S01 CD8 T cell state was done with ToppFun. Significant GO: Molecular Function pathways were selected based on the enrichment of the top 20 cell state-associated genes. Top pathways were rank-ordered by their −log 10 FDR corrected p-values.

### CytoTRACE Developmental Status

The CytoTRACE tool (v 0.3.3) was used to determine the developmental status of malignant cells in the scRNA-seq expression dataset. The scRNA-seq expression matrix was normalized to CPM and run with Scanorama batch correction according to the steps listed in the Custom Integrated CytoTRACE tutorial.

### EUS-FNB Pseudo-Bulk Deconvolution and Ecotype Recovery

To create a pseudo-bulk expression for each sample, all transcripts for each gene were summed across all cells for each EUS-FNB sample.

The resulting expression matrix was then normalized in the same fashion as the publicly available bulk RNA-seq expression datasets as described in the Bulk expression deconvolution method section.

EcoTyper ecotypes and cell states were computed for the pseudo-bulk expression matrix using an identical methodology to EcoTyper cell state recovery on the clinical bulk expression datasets, as described in the EcoTyper ecotype discovery method section.

### Results

Quantification of Malignant and Tumor Microenvironment Cell States in Integrated scRNA-Seq Datasets

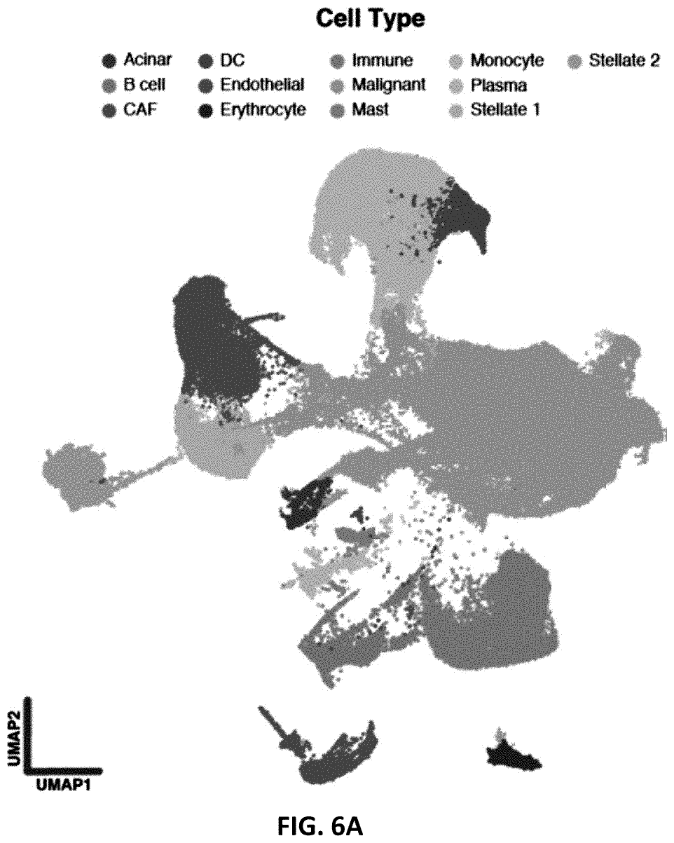

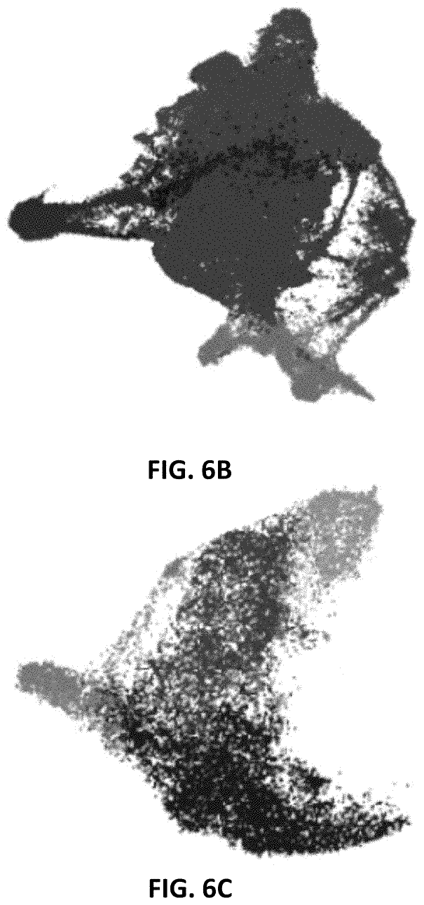

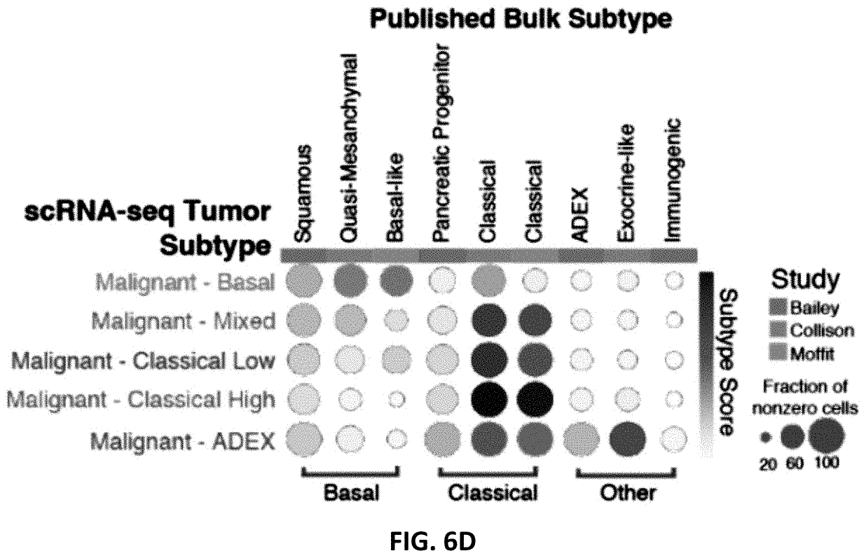

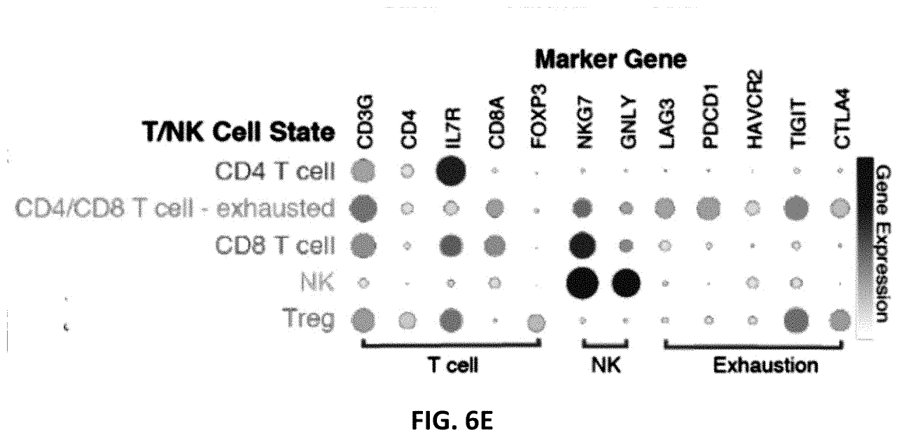

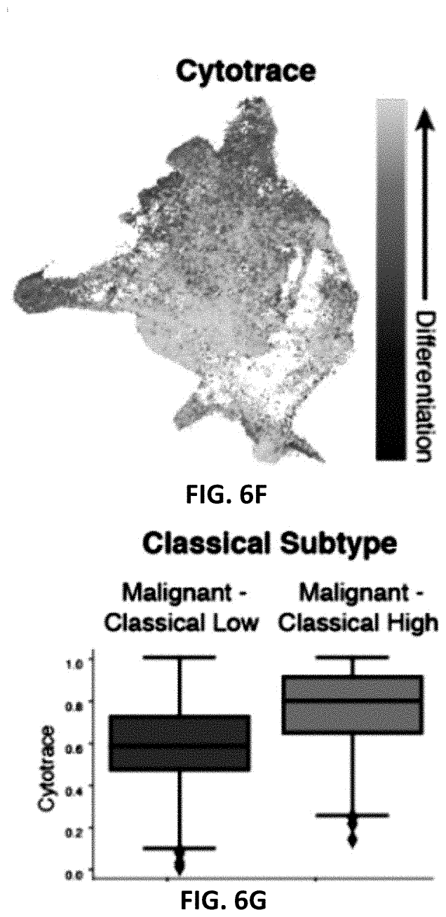

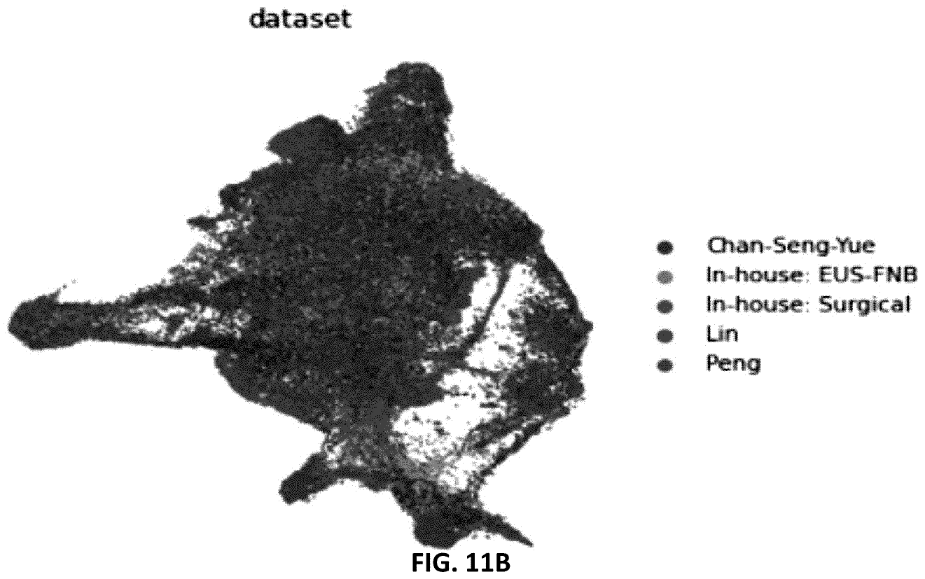

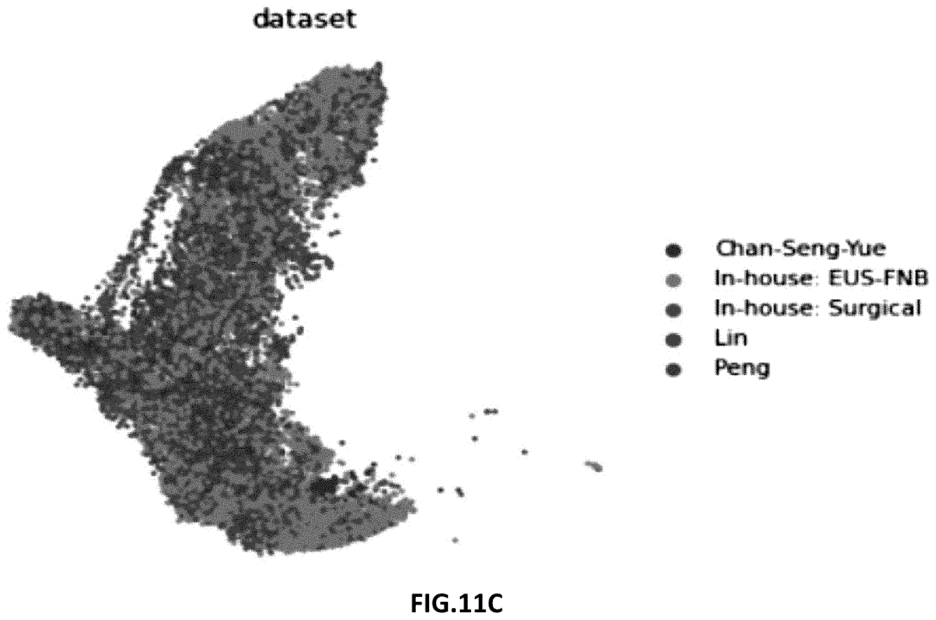

scRNA-seq of PDAC from standard endoscopic ultrasound-guided fine needle biopsy (EUS-FNB) specimens was performed at the time of diagnosis and from surgical samples obtained from tumor resections to enable a comprehensive view of PDAC. In total, 28 k cells were acquired across 22 samples for our in-house EUS-FNB cohort, and 20 k cells were acquired from 6 samples for the in-house surgical cohort. To increase power, the in-house scRNA-seq data was then combined with three publicly available datasets: Peng et al, Lin et al., and Chan-Seng-Yue et al. Following the integration of the single-cell data with Seurat, the sample size was increased to a total of 141 k cells from 77 samples. The integrated dataset was clustered and annotated based on known cell type markers, where a total of 13 cell types were found (FIG. 6A). With the exception of the erythrocytes, every cluster had representation from multiple datasets, indicating that significant dataset-specific batch effects were largely removed during integration (FIG. 11A).

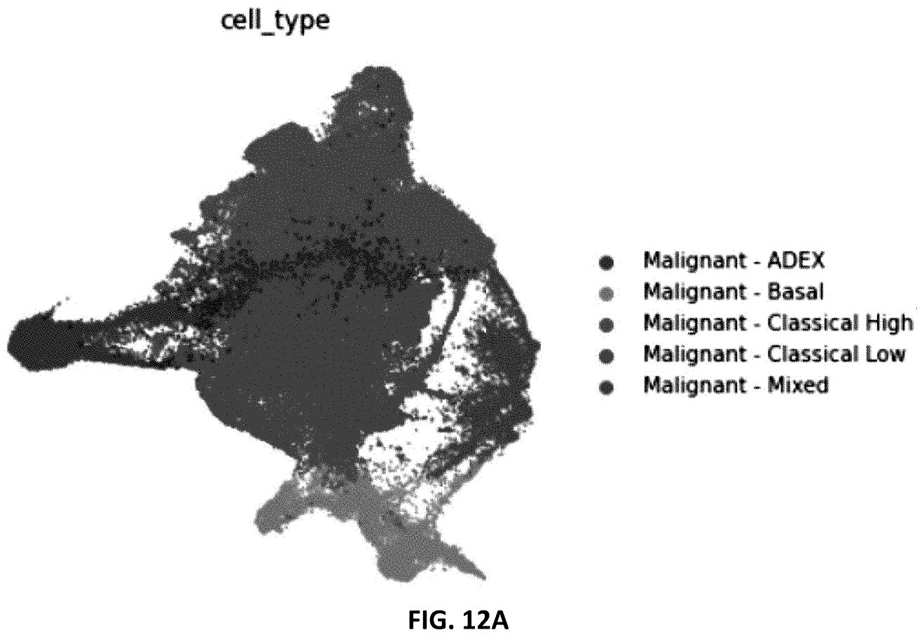

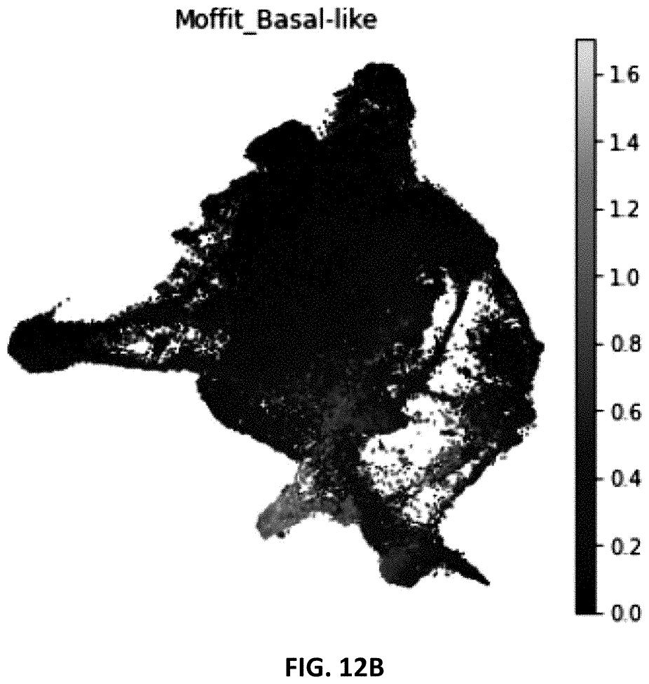

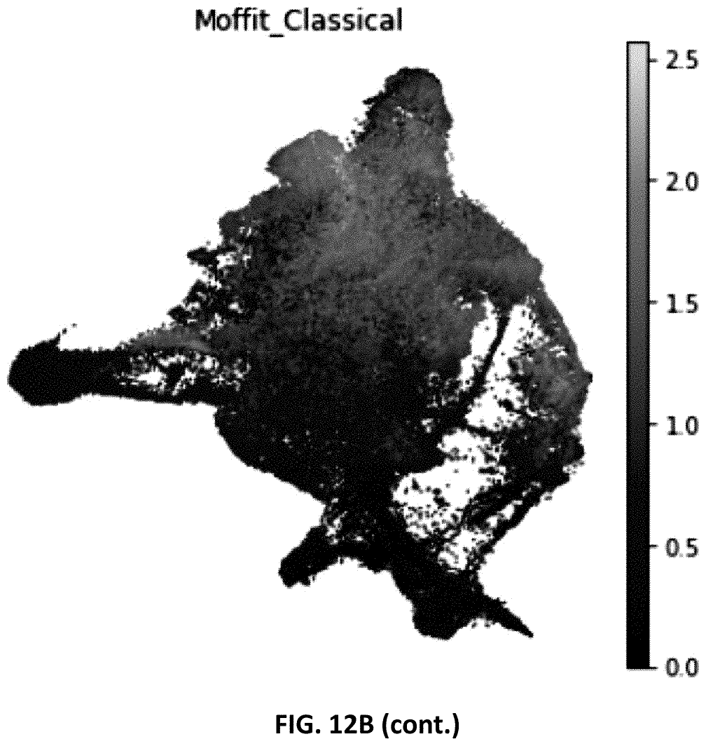

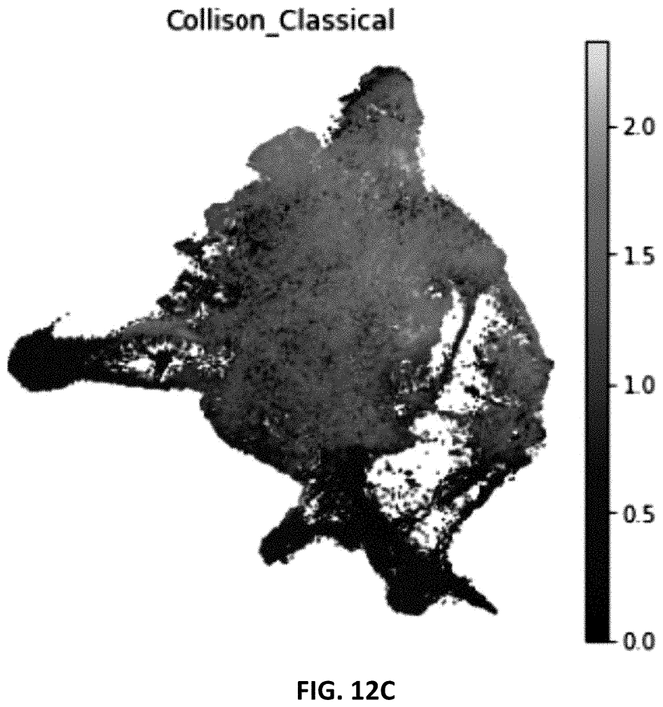

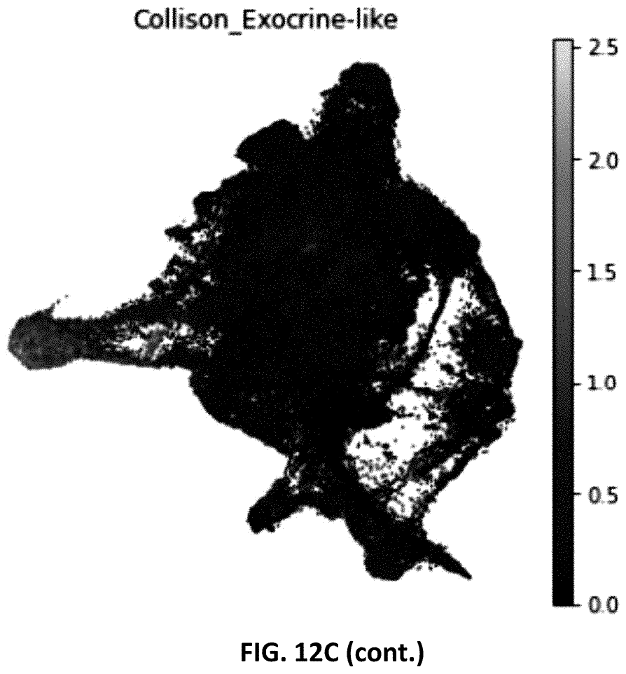

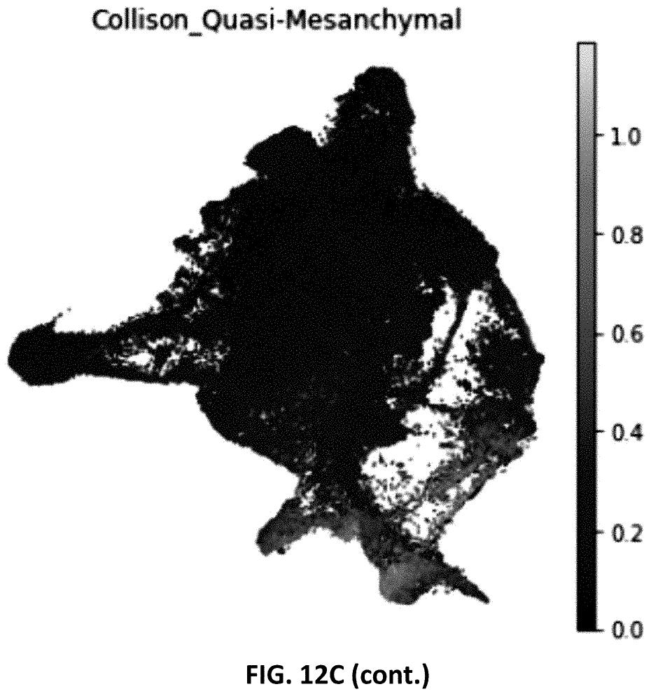

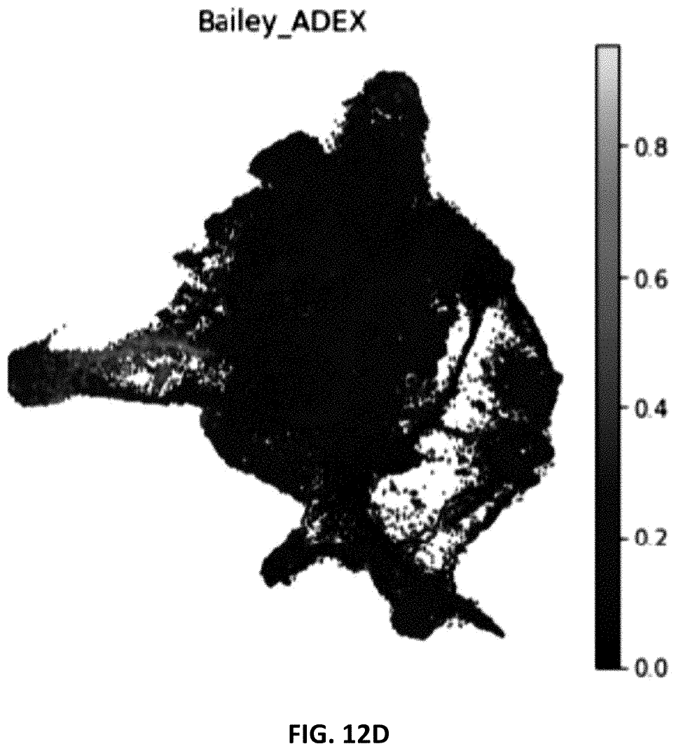

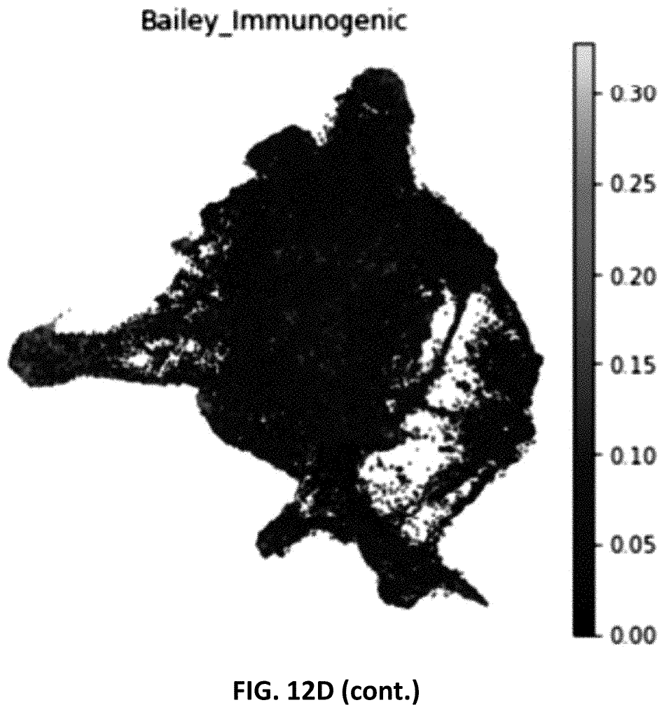

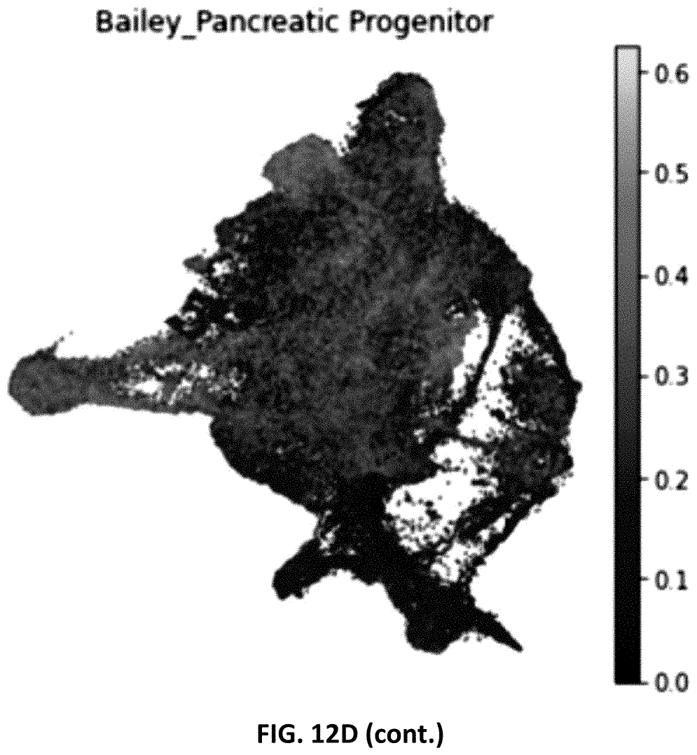

The Malignant and T cell clusters were further clustered and subdivided into more fine-grained cell states. For the malignant cluster, five total subclusters were identified (FIG. 6B). These subclusters were categorized based on the aggregate expression of markers from three previous studies: Bailey et al., Collison et al., and Moffit et al (FIG. 6D). When the expression signatures derived based on these known subtypes are overlain on the malignant cell subclusters, remarkably good concordance is found with the known subtypes (FIGS. 12A, 12B, 12C, and 12D). Using gene expression microarray data, Moffitt et. al. categorized PDAC into classical and basal-like populations. Bailey and Collision later used bulk sequencing data to describe squamous, endocrine, pancreatic progenitor, and immunogenic (Bailey) and quasi-mesenchymal, classical, and ADEX (Collison) subtypes of PDAC. The main consensus subtypes found in these studies are 1) Classical (Classical, pancreatic progenitor), and 2) Basal (basal, squamous-like, quasi-mesenchymal). Some studies have called the existence of the immunogenic and ADEX/endocrine subtypes into question, however, in addition to Basal and Classical subtypes, we do also see a cluster of overexpressing genes associated with the ADEX/endocrine subtype was also seen in the single cell data. Based on concordance with the known subtypes, we annotate the five malignant subclusters are annotated as follows: Basal, Mixed, Classical Low, Classical High, and ADEX. In addition to further clustering the malignant cells, the T lymphocyte cluster is further subdivided into five states: CD4 T cell, CD8 T cell, CD4/CD8 T cell—exhausted, NK, and Treg (FIGS. 6C and 6E). For all the above cell states, good representation is seen from multiple datasets, indicating none of the clusters are an artifact of a particular dataset. (FIGS. 11B and 11C).

The developmental status of the malignant subclusters was then determined. For this, the tool CytoTRACE was used to obtain developmental scores for each tumor cell, with cells having a high CytoTRACE score being less developmentally mature, and those with a low score being more differentiated. It was found that two of the five malignant subclusters, ADEX and Classical High, are more differentiated than the other subclusters (FIGS. 6F and 6G). This developmental continuum between Classical and Basal clusters has been previously shown, as the Basal subtype is known to be more likely to undergo the epithelial-mesenchymal transition (EMT), resulting in higher rates of metastasis. However, it is interesting that the “less classical” of the two classical subclusters is more poorly differentiated than the other, perhaps indicating that this subcluster carries more stem-like features, and as a result, is more aggressive than its more developmentally mature counterpart.

### Community Identification in Clinical Bulk Expression Datasets

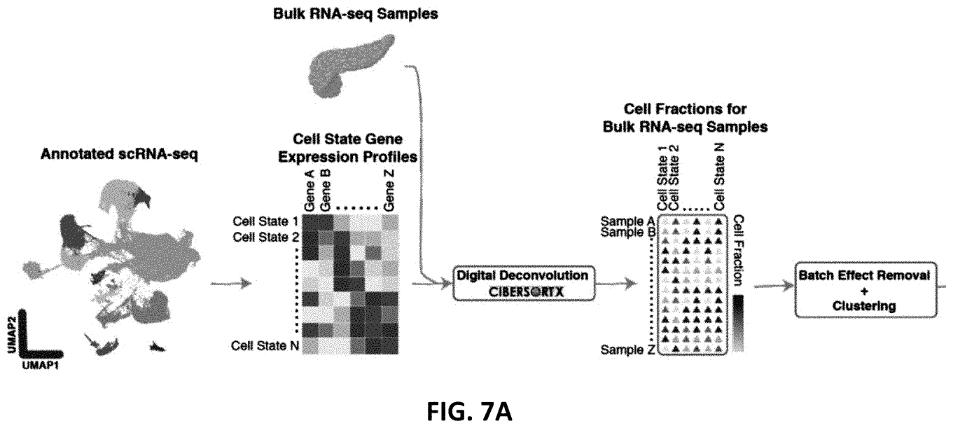

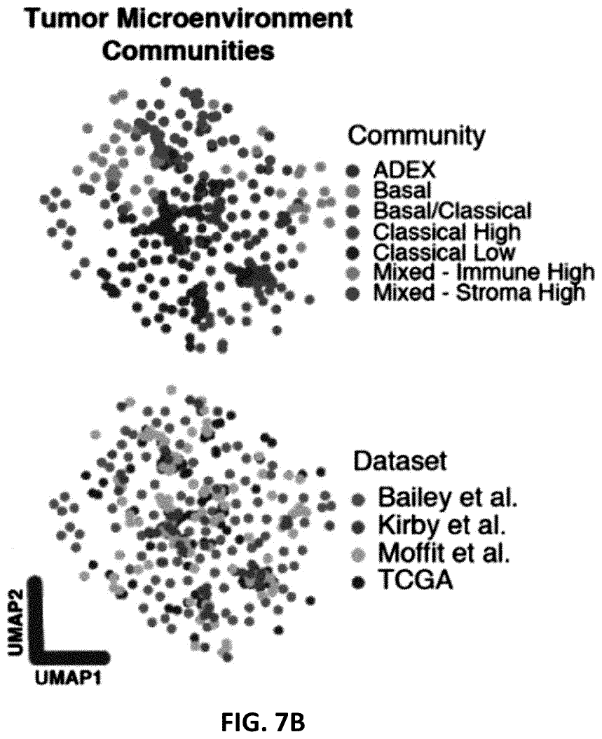

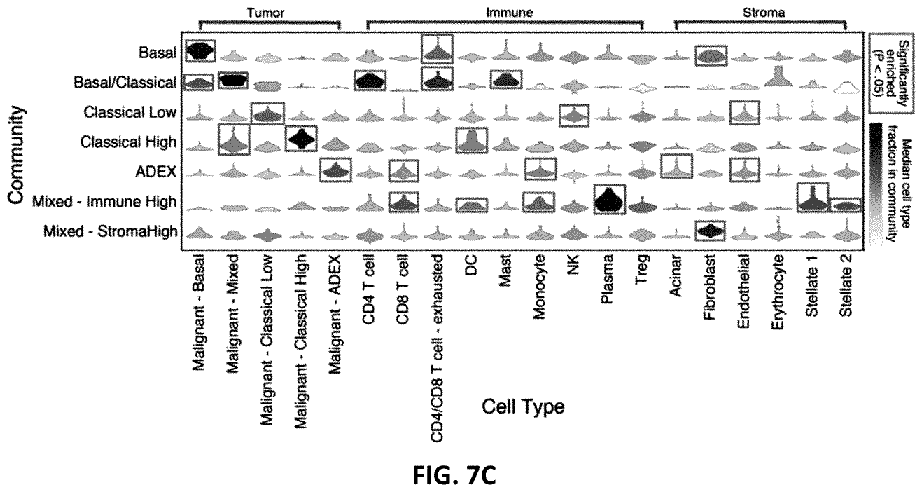

The single-cell expression profiles were then extended to publicly available bulk RNA-seq datasets with associated clinical metadata. To this end, the digital deconvolution tool CIBERSORTx was applied to obtain cell type proportions for each bulk RNA-seq sample (FIG. 7A). First, a signature matrix was derived from expression profiles of each annotated cell type in the single cell RNA-seq dataset. Then, using CIBERSORTx, 391 samples were deconvolved from the four separate bulk RNA-seq datasets: Moffit et al., Bailey et al., Kirby et al., and Collison et al. After imputed cell fractions were computed, clusters of samples with similar cell fraction profiles were identified. These clusters of samples are termed “communities”. After batch-correcting the sample cell fractions to account for differences in data type and methodology between the bulk datasets, the samples were clustered with the graph-based Leiden clustering algorithm. Following clustering, seven distinct communities were left, each with representation from at least two of the bulk RNA-seq datasets (FIG. 7B). The communities were then labeled based on patterns of cell fraction enrichment (FIG. 7C). Specifically, four communities fell on a spectrum between the main basal and classical PDAC subtypes. These communities were termed Basal, Basal/Classical, Classical Low, and Classical High. A community enriched with the ADEX malignant subtype cell fraction was also found, as well as two communities with higher proportions of stromal/immune cells, which were named Mixed-Stroma High and Mixed-Immune High. Interestingly, an overrepresentation of exhausted T cells and the Basal and Basal/Classical communities are seen (FIG. 7H).

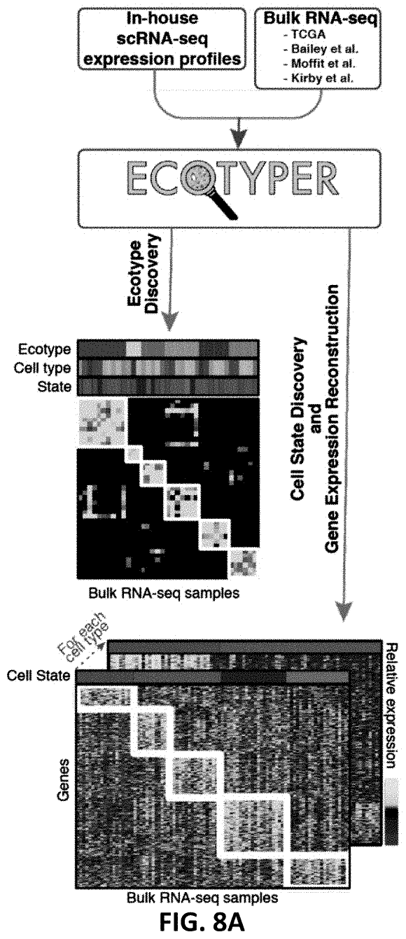

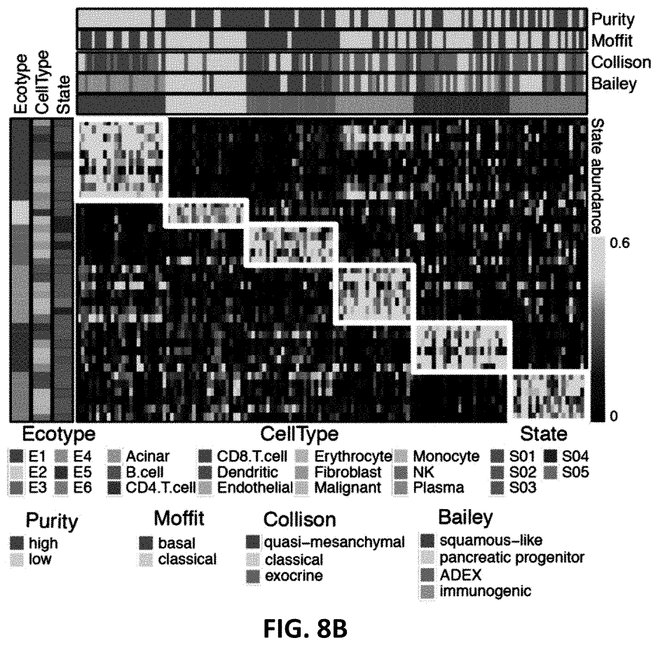

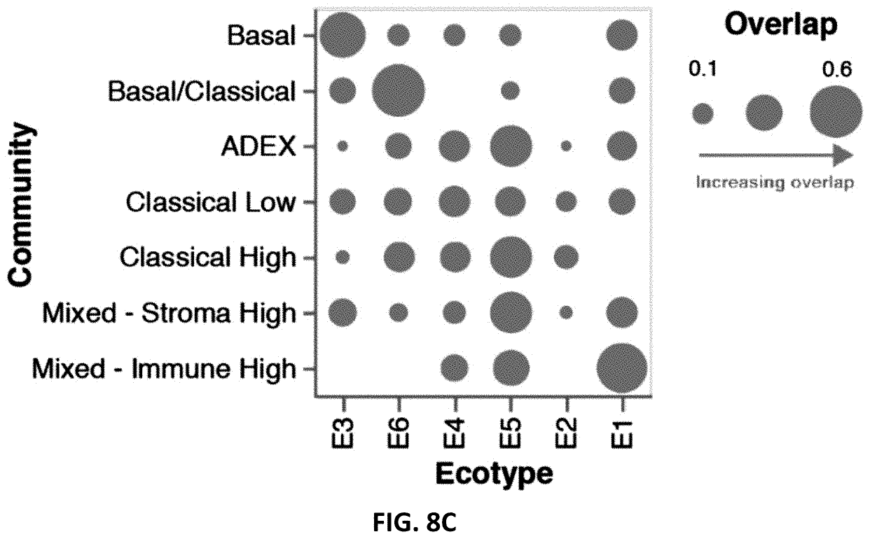

The discovered communities were then orthogonally validated with the TME dissection tool EcoTyper. Similar to the unsupervised community detection approach, EcoTyper takes as input single-cell expression profiles. These profiles are then used to impute gene expression and identify cell state for samples in bulk RNA-seq and microarray datasets (FIG. 8A). Additionally, EcoTyper groups significantly associated cell states into Ecotypes, which are an analogous abstraction to the TME communities. When EcoTyper was applied to the 391 samples from the bulk expression datasets, six distinct ecotypes were found, labeled E1-E6, each with distinct patterns of cell state enrichment (FIG. 8B). Ecotype E1 is highly enriched for immune cell states, as it has low tumor purity, is composed of mainly immune cells states and displays high levels of enrichment for bailey immunogenic samples, which are samples known to have high ratios of immune cells. Further indicating an immune-enriched makeup, ecotype E1 also shows high overlap with the Mixed-Immune High community (FIG. 8C). In addition to EcoTyper E1, ecotypes E3 and E6 show high overlap with the Basal and Basal/Classical communities respectively, while ecotypes E2, E4, and E5 do not show high association with any of the identified communities.

### Patterns of TME Cell State Association are Indicative of Patient Outcome

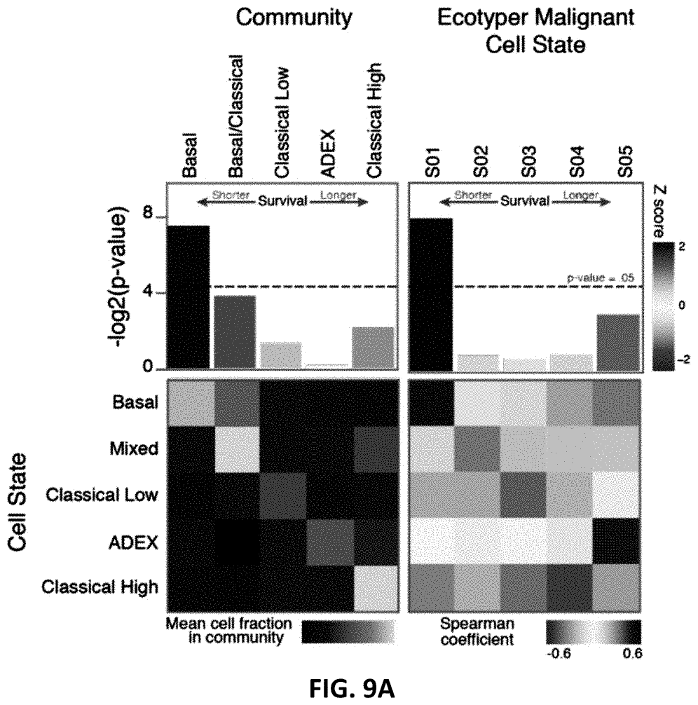

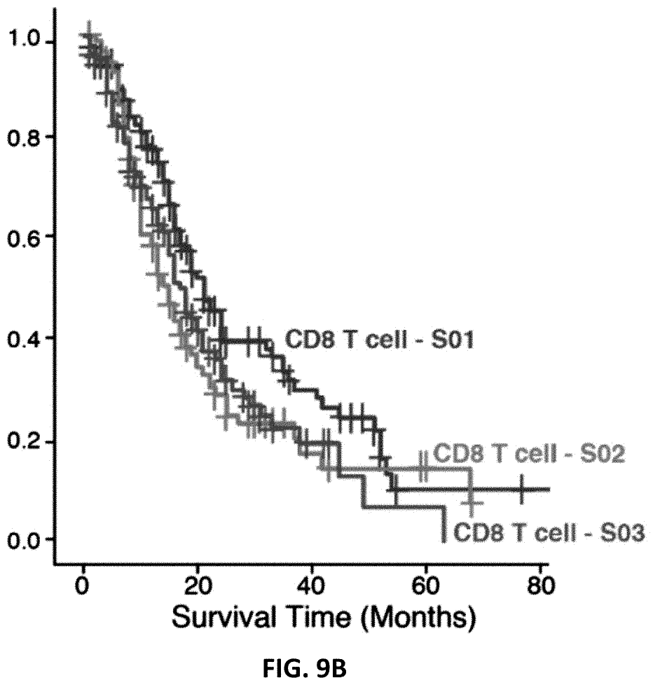

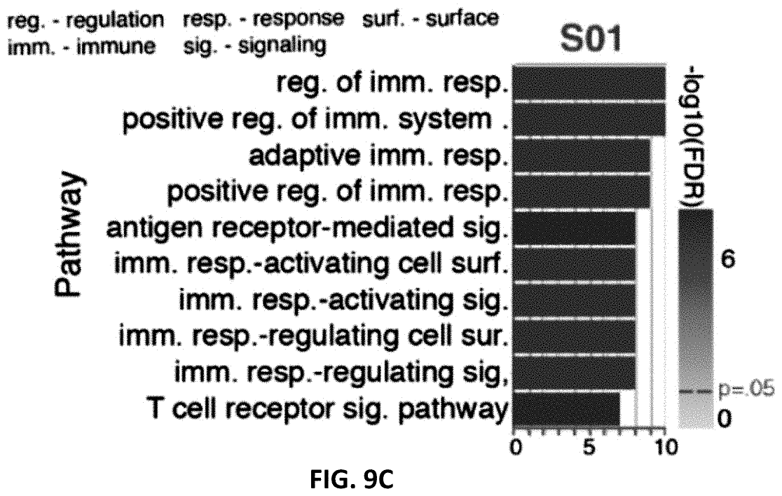

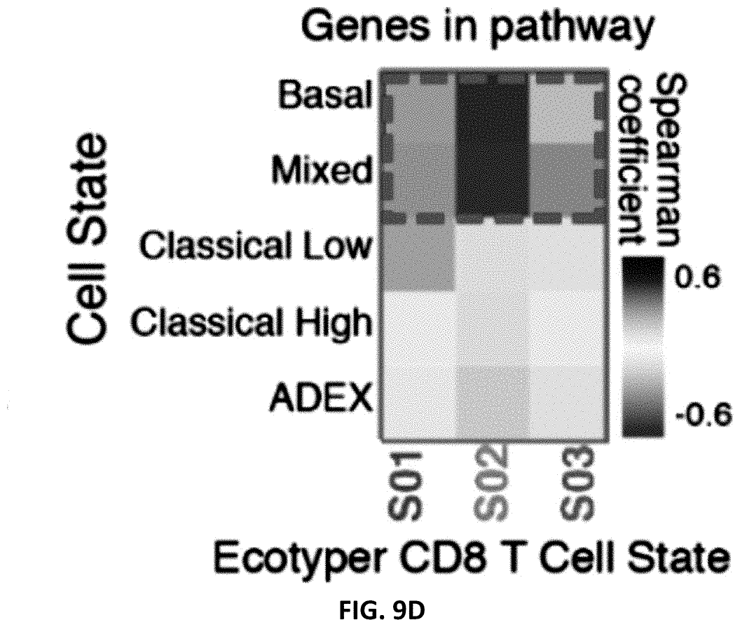

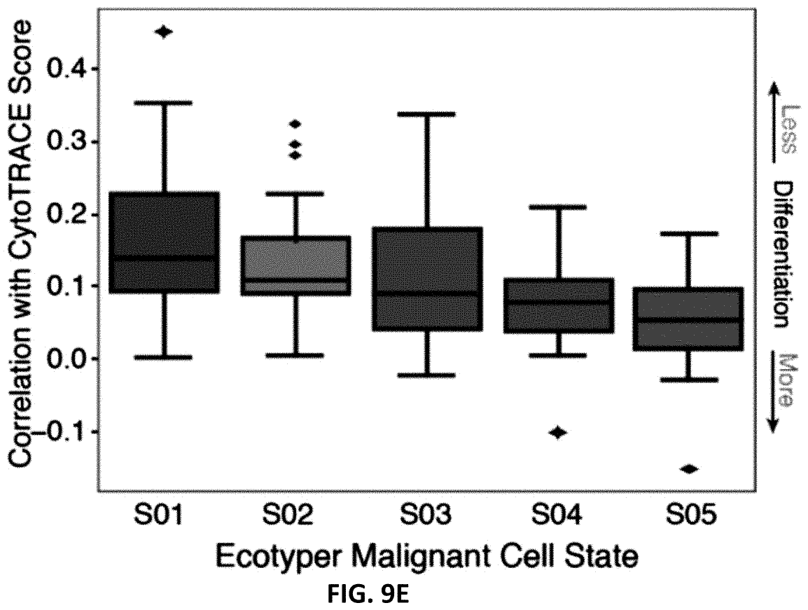

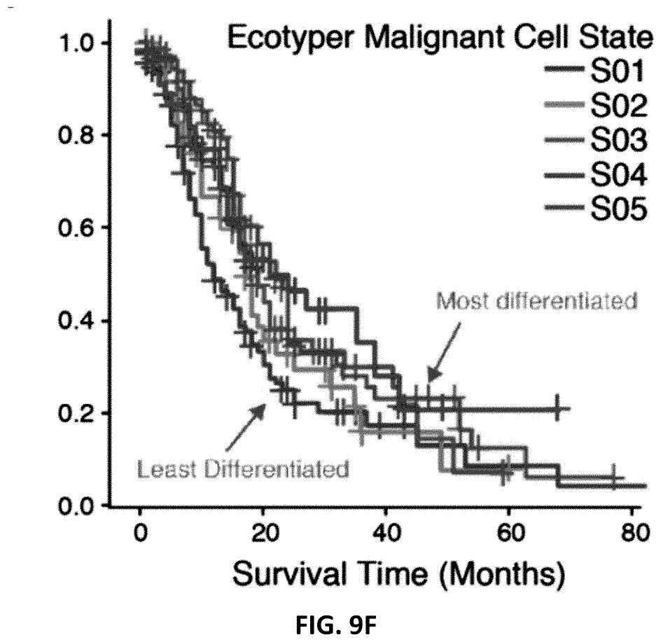

Made possible due to the availability of clinical metadata for the bulk datasets, survival analysis was performed on the cell states that make up the above communities and ecotypes. As expected, there is a spectrum of survival related to the abundance of malignant subtypes, with the most basal cell states showing the poorest survival, while the most classical displayed the greatest overall survival (FIG. 9A). Meanwhile, intermediary/mixed subtypes fall between the two. This relationship between basal and classical subtypes has been described extensively in the literature. This trend appears in both the deconvolved cell state fractions comprising the TME communities, as well as EcoTyper discovered cell states, where a correlation is seen between cell state fractions and EcoTyper malignant cell state abundances. Of particular note, it was also shown that the Classical Low cell state has worse survival than the Classical High cell state, indicating that the Classical subtype partitions into survival distinct groupings, consistent with findings by. Additionally, it was found that EcoTyper discovered CD8 T cell states are associated with survival, where the S01 CD8 T cell state is indicative of improved overall survival, while its S02 and S03 counterparts portend worse outcomes (FIG. 9B). To identify pathways upregulated in S01, a gene set enrichment analysis (GSEA) was performed on the top 20 genes significantly associated with the S01 CD8 T cell state. It was found that S01 is enriched for genes associated with positive regulation of immune response, suggesting that tumors with more activated CD8 T cells have better outcomes (FIG. 9C). Next, the abundance of the EcoTyper discovered CD8 T cell states were correlated with the deconvolved malignant cell state fractions to see if the activated S01 CD8 T cell state was correlated with a particular malignant subtype. It was found that the immune-active S01 CD8 T cell state is negatively correlated with the Basal and Basal/Classical cell states, while its counterparts S02 and S03 are positively associated with the basal subtypes (FIG. 9D). These findings suggest that the more aggressive, EMT-like Basal and Basal/Classical communities have less activated CD8 T cells present in their TME than other subtypes.

Next, the developmental statuses of the EcoTyper-discovered malignant cell states were compared. To do so, CytoTRACE score correlations were taken from the malignant single-cell data for the top 20 genes associated with each malignant cell state, and their distribution was displayed across each EcoTyper malignant cell state. It is found that there not only exists a survival continuum between malignant cell states but also a developmental continuum, with Basal being the least differentiated and Classical High the most (FIGS. 9E and 9F). This finding aligns with the developmental analysis in the single-cell data, where developmental differences from cells across different subtypes were found. Survival analysis was also performed where each sample was partitioned into its most abundant EcoTyper malignant cell state, where it was found that the developmental continuum also extends to survival, with the least developed malignant cell state having the worst overall survival.

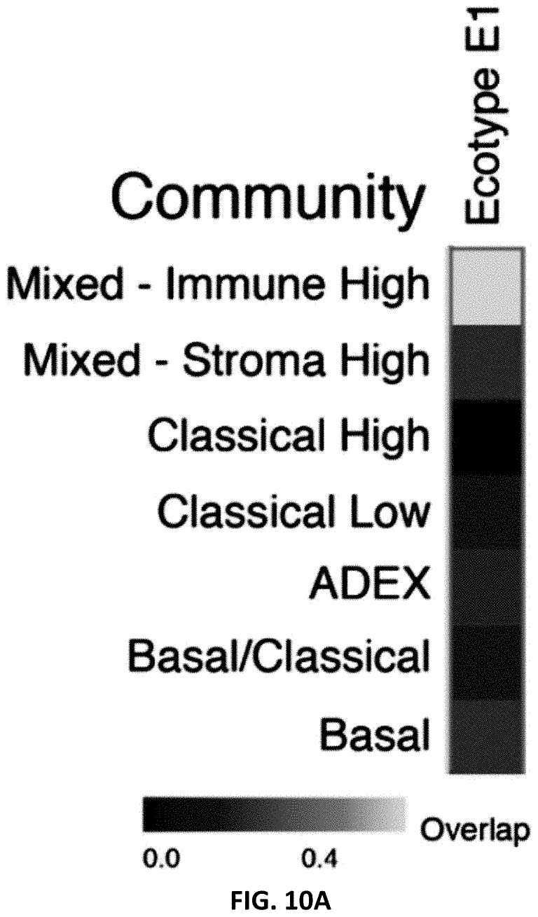

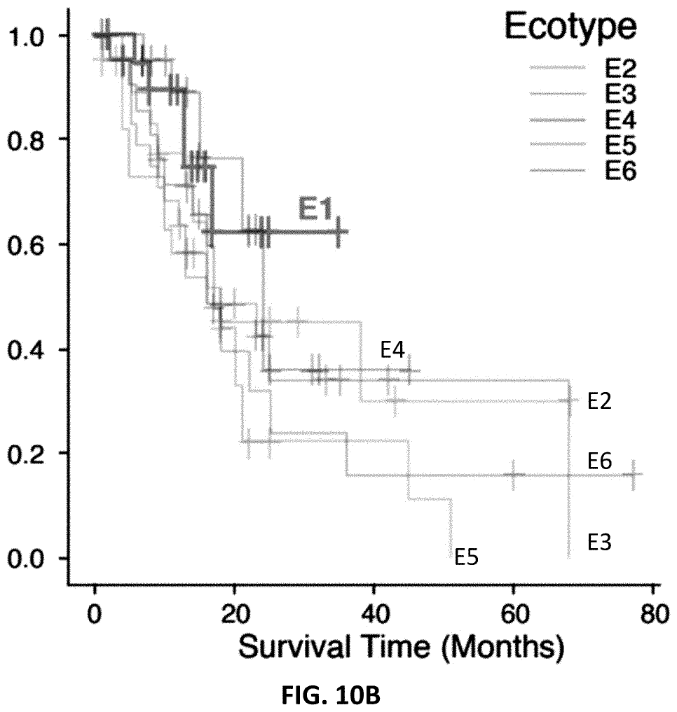

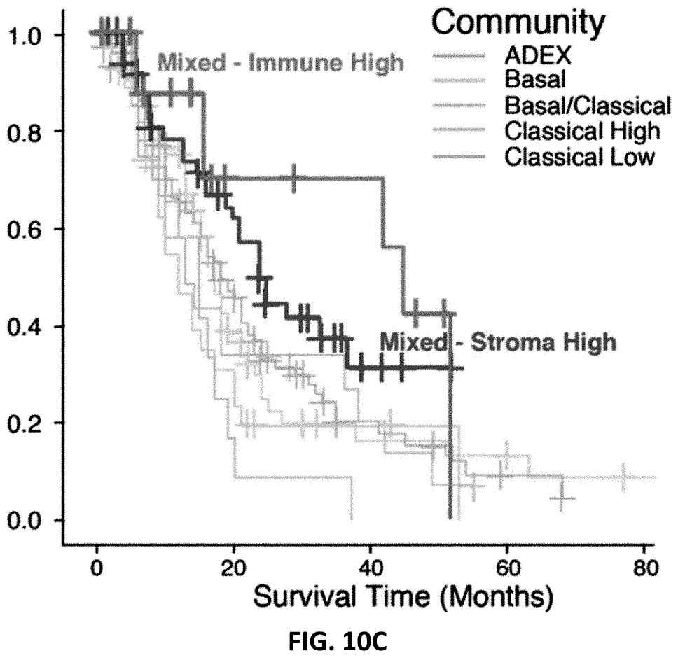

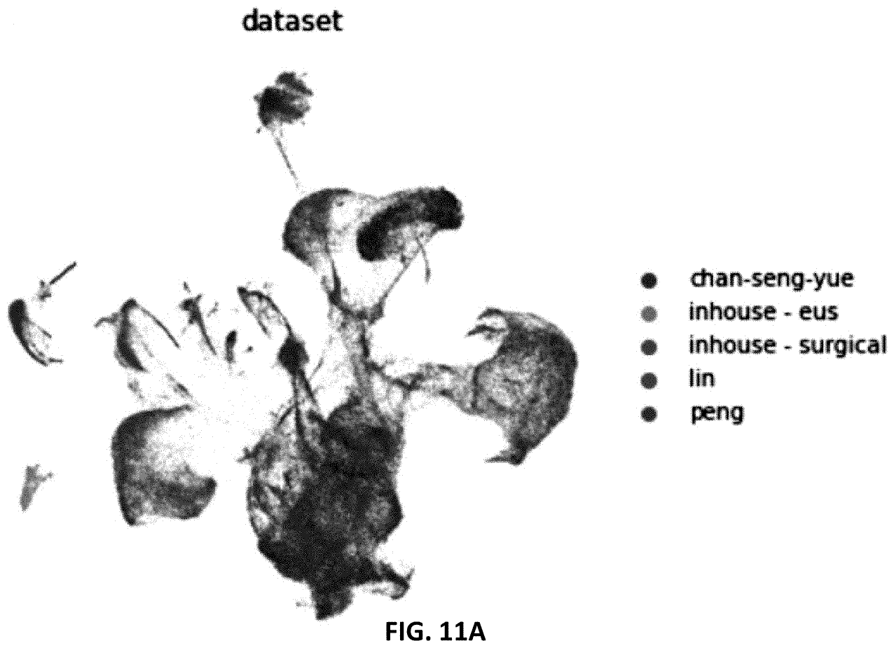

Survival associated at the community and ecotype level was also found, where stromal and immune-dominated ecotypes are associated with improved survival. In particular, TME patterns with enriched immune cells such as the ecotype E1 and the Mixed—Immune High community show improved survival over the tumor-dominated communities and ecotypes (FIGS. 10A and 10B). Additionally, the Mixed—Stromal High community that was enriched for various stromal states also showed increased survival compared to tumor-centric communities and ecotypes (FIG. 10C). These findings align with previous studies showing increased immune and stromal infiltration is known to portend better outcomes in a variety of cancer types.

TME Cell State Associations in Pseudo-Bulked EUS FNB Cohort

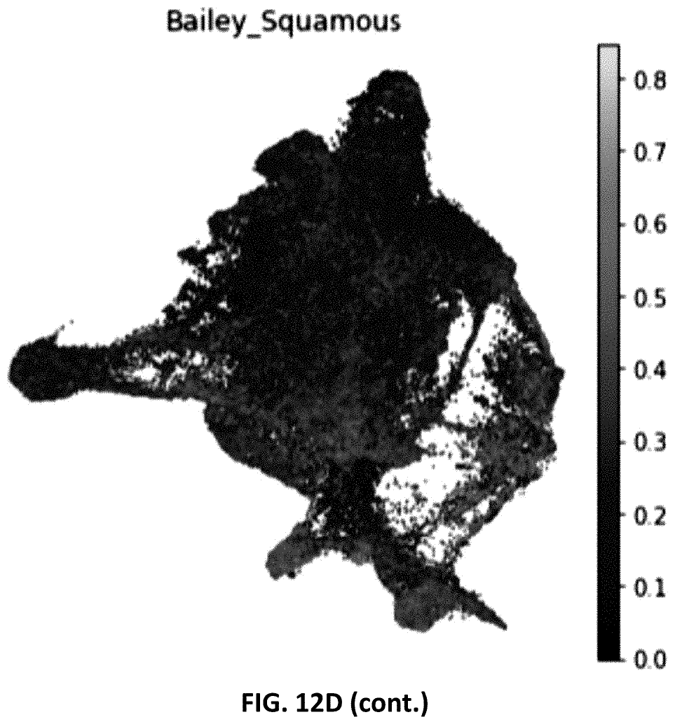

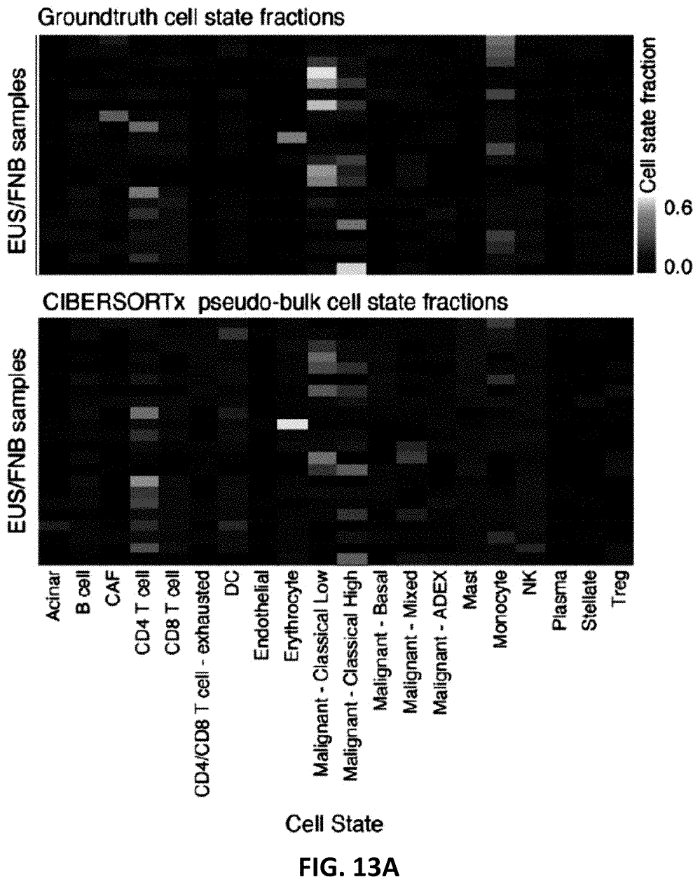

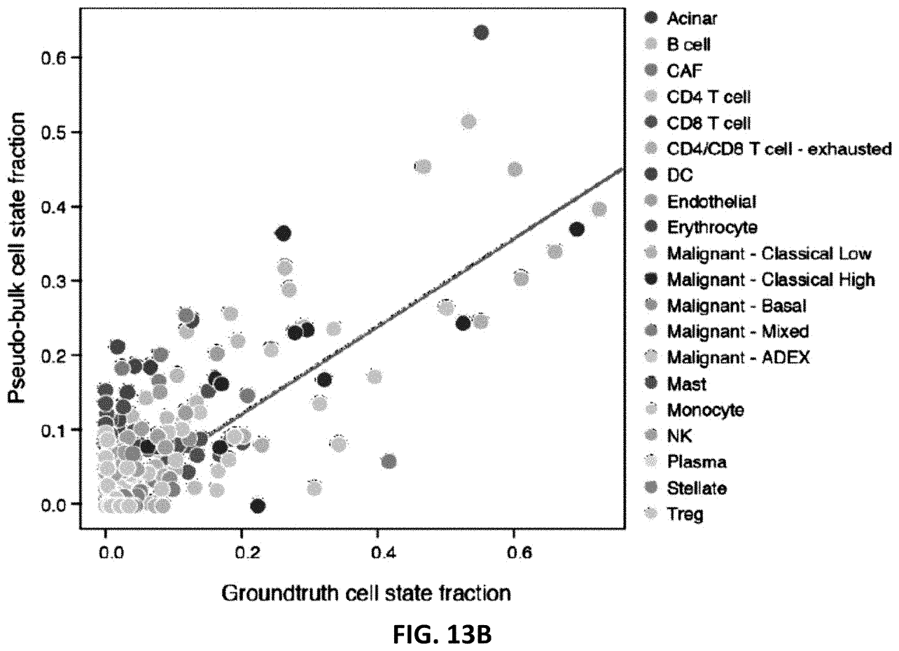

Finally, due to the potential clinical utility of EUS-FNB bulk RNA-seq samples, it was determined whether patterns of cell state abundance found in the clinical bulk expression datasets were also present in the EUS-FNB cohort. In lieu of the absence of bulk RNA-seq data for our EUS-FNB biopsy cores, a pseudo-bulked mixture of our scRNA-seq EUS-FNB samples was created. The resulting mixture was then deconvolved with CIBERSORTx to imputed cell state fractions, and used as input to EcoTyper to generate abundances for EcoTyper cell states discovered in the bulk expression datasets. To benchmark the pseudo-bulk deconvolution, ground-truth cell type fractions (calculated from manually annotated single-cell data) and CIBERSORTx deconvolved cell fractions were compared, and the pseudo-bulk deconvolved and ground-truth cell state fractions were highly correlated (spearman coefficient=0.53, p-value<0.005) (FIGS. 13A, 13B, and 13C).

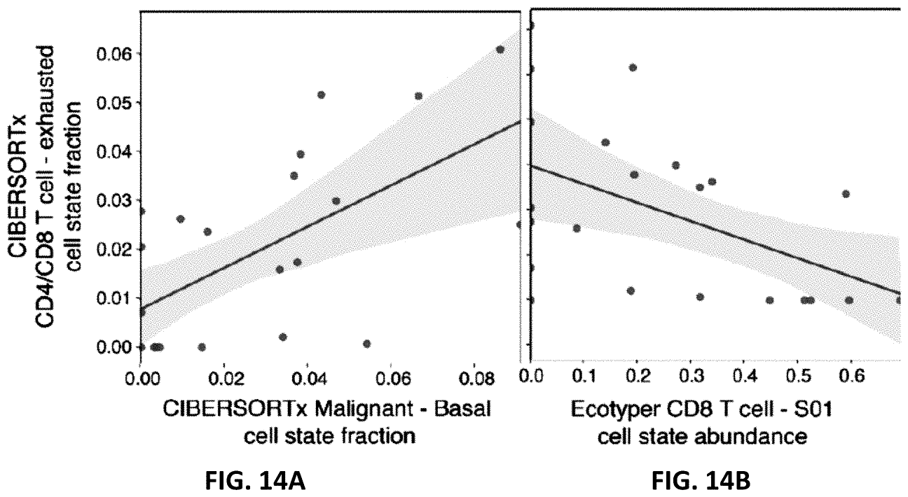

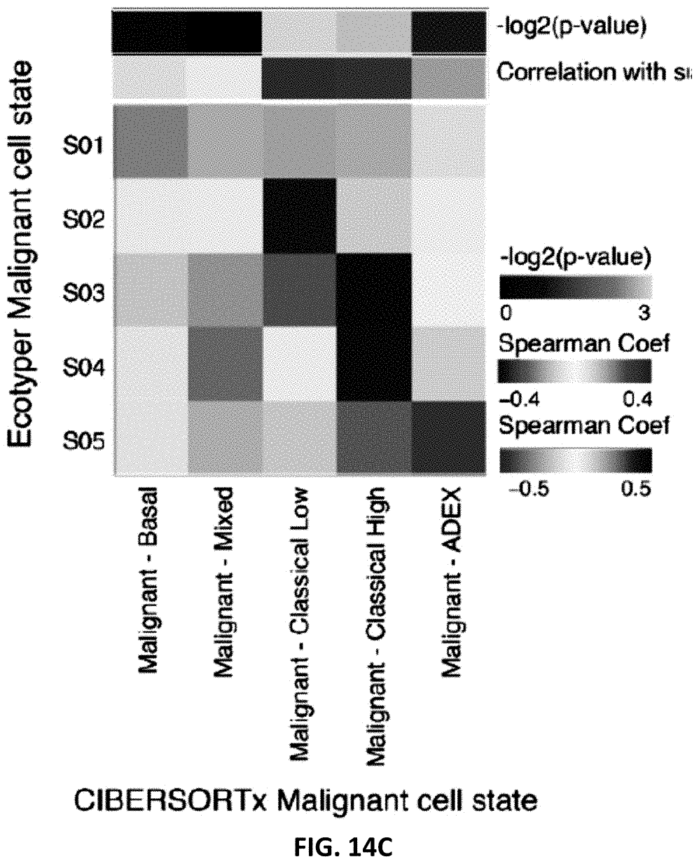

In bulk expression datasets it was found that the abundance of the more aggressive basal subtype was associated with the presence of exhausted CD4/CD8 T cells. A similar trend is seen in the EUS-FNB TME, with the exhausted CD4/CD8 T cell and Malignant Basal cell fractions being highly correlated (spearman coefficient=0.58, p-value<0.005) (FIG. 14A). The abundance of exhausted CD4/CD8 T cells were also negatively correlated with the EcoTyper discovered immune-activated S01 CD8 T cell state (spearman coefficient=−0.53, p-value<0.05, FIG. 14B). Additionally, the CIBERSORTx deconvolved cell states remain correlated to EcoTyper-discovered malignant cell states in a similar fashion to the bulk expression clinical datasets (FIG. 14C). The malignant subtype cell state abundances also show similar trends in terms of survival, with more basal subtypes showing worse outcomes than classical subtypes. However, the ADEX subtype shows the worst survival of all subtypes, which is inconsistent with survival trends in the bulk expression datasets, and none of these associations are significant—possibly due to the small cohort size (n=22).

### Discussion

Using gene expression microarray data, Moffitt et. al. categorized PDAC into a classical and basal-like population. Bailey and colleagues later used bulk sequencing data to describe squamous, endocrine, pancreatic progenitor, and immunogenic subtypes of PDAC. Several of these subtypes have been called into question, and the current consensus PDAC subtypes remain squamous/basal-like and classical/pancreatic progenitor.

By analyzing scRNA-seq datasets comprised of in-house time-of-diagnosis EUS-FNB biopsies and surgical specimens, along with publicly available datasets, these subtypes were not only recapitulated (classical, squamous-like, and ADEX), but also mixtures of subtypes (Mixed Basal/Classical) were also recapitulated as has been described herein. Further, by gene marker sets from previous studies, low and high classical malignant cell states were identified, which contained a dichotomy of less-developed/stem-like and more-developed classical tumor cell states that were identified with CytoTRACE. Using digital tissue deconvolution of publically available bulk expression datasets with CIBERSORTx, cell fractions for samples in these datasets were inferred. With unsupervised clustering, sample communities that were associated with clinical survival were identified. These communities were also orthogonally validated with the TME discovery tool EcoTyper. Consistent with prior data, the basal community conferred a significantly worse prognosis as compared to the most classical community. Interestingly, other communities fell on a continuum between these two populations, and their ordering was based on factors such as TME makeup and malignant subtype. Additionally, this ordering also correlated with developmental status, indicating that the communities associated with poorer survival are also more EMT-like. It is also found that samples with increased immune and stromal fractions are associated with improved overall outcomes.

While scRNA-seq is expensive and technically challenging to perform at the time of diagnosis, bulk RNA-seq is much less expensive and far more practical. The results were thus extended to readily available bulk gene expression data and a pseudo-bulk RNA-seq expression mixture from the EUS-FNB cohort by applying CIBERSORTx deconvolution and EcoTyper ecotype and cell state discovery. Furthermore, the ability to identify distinct developmental states within the luminal progenitor compartment of breast cancer using CytoTRACE was demonstrated, where knocking down genes associated with the immature malignant cell state led to decreased tumor growth in vivo. It is proposed that similar methods could be applied to PDAC by targeting molecular pathways specific to the ED-classical tumor cell state, which could significantly improve clinical outcomes for these otherwise high-risk patients.

The methodology outlined lends itself to both the discoveries of novel cell types/states/signatures, patient prognosis, and treatment paradigms. When utilizing bulk sequencing and NGS techniques for discovery, rare cell types and variants can be missed due to inherent coverage limitations with bulk sequencing. Unlike NGS, scRNA-seq allows one to individually profile each cell, and thus appreciate the full breadth of diversity in cell types and profiles within the tumor microenvironment (TME). This is especially applicable in cancers like PDAC, where only ˜⅕ of cells in a tumor biopsy are PDAC tumor cells, with the vast majority of cells comprising various components of the TME. The ability to clinically validate findings in scRNA-seq using digital deconvolution methods allows NGS data to be mined with previously validated profiles, and thus a prognostic signature can be identified.

Unfortunately, for PDAC, multiple avenues of targeted therapy have previously failed large clinical trials. Drugs targeting the tumor stroma, KRAS, EGFR, and VEGF, that had previously been validated using in-vitro as well as in vivo murine models, have unfortunately failed phase III trials. It is clear that results seen in murine models often do not recapitulate those seen in patients. It is proposed that scRNA-seq signatures seen in mouse or treatment-responding patients be used to deconvolute bulk sequencing from biopsies of patients receiving treatment, to give a real-time signature if the treatment is working as expected. This could help give an early signal if things are behaving as predicted, and if not, why (i.e. are certain cell types in the TME over or under-represented, and could this account for the clinical response—or lack thereof— seen?). In addition, one could use this technology to define a strict cut point of expression in a given cell type in order to better classify patients for trials. For instance, for PEGPH20, high hyaluronic acid staining was used as an inclusion in the trial of PEGPH20 in stage IV PDAC. This data can be biased by which paraffin-embedded section is looked at and stained, and is often very approximate in nature. scRNA-seq allows for one or multiple exact cut points gleaned from a large number of cells that are looked at in a non-biased way. Cut points validated could then be applied to digitally deconvoluted data from bulk sequencing done on potential trial participants, allowing for more rigorous and exact definitions, and inclusion/exclusion criteria. This may allow for an enhanced likelihood of trial success, and decreased likelihood of data misinterpretation.

It is acknowledged that the total number of patients in the experimental subset (N=13**with added), and the number of total cells (N=##) is less than the limited prior work looking at scRNA-seq data in human PDAC, but this analysis has multiple strengths. For one, this work is the first to use time of diagnosis EUS-FNB PDAC specimens for use in sc-RNAseq—thus eliminating any treatment or time from diagnosis bias in the data. In addition, differing from prior work, the scRNA-seq findings are tested in an independent cohort of surgically resected PDAC patients, and the findings are clinically validated in previously published large PDAC datasets.

In summary, cells were reliably isolated from pancreatic cancer time-of-diagnosis EUS-FNB specimens for scRNA-seq. CytoTRACE was utilized to identify tumor cell states predictive for survival, specifically elucidating a novel developmental state dichotomy within classical PDAC, and corroborating the previously described poorly prognostic squamous-like subtype. The scRNA-seq and CytoTRACE results were next tested using large published PDAC datasets by applying CIBERSORTx digital tissue deconvolution paired with unsupervised clustering and EcoTyper. The innovative methodology provides unique insight and has the potential to improve clinical decision-making through better upfront risk stratification.

What is claimed is:

1. A computer-implemented method of selecting a treatment for pancreatic adenocarcinoma (PDAC) in a patient in need, the method comprising:
a. receiving, at a computing device, a single-cell RNA sequencing (scRNA-seq) dataset comprising at least one RNA expression signature and associated cell state and a bulk RNA sequencing sample derived from a tumor sample obtained from the patient;
b. transforming, using the computing device, the bulk RNA sequencing sample into a cell state fraction dataset comprising a plurality of cell states and associated proportion of the expression of the scRNA-seq dataset attributable to each cell state;
c. assigning, using the computing device, one tumor microenvironment (TME) cell state from a TME dataset to the patient based on the cell fraction dataset; and
d. selecting a treatment for the patient based on the assigned TME cell state.

2. The method of claim 1, wherein each TME cell state of the TME dataset comprises a unique distribution of cell fractions among the plurality of cell state categories.

3. The method of claim 2, wherein each TME cell state further comprises a predicted clinical outcome associated with each TME cell state.

4. The method of claim 3, further comprising producing a TME dataset by:
a. receiving, at the computing site, a plurality of calibration single-cell RNA sequencing (scRNA-seq) datasets and associated clinical outcome measurements obtained from at least one PDAC patient population;
b. transforming, using the computing device, each calibration scRNA-seq dataset into a calibration cell fraction dataset comprising a plurality of cell states and associated proportion of the expression of the scRNA-seq dataset attributable to each cell state;
c. assigning, using the computing device, the plurality of calibration cell fraction datasets to a TME cell state of the TME dataset, wherein the TME cell state comprises a cell fraction distribution shared by all calibration cell fraction datasets assigned to the TME cell state; and
d. associating, using the computing device, a clinical outcome to the TME cell state, the clinical outcome comprising the shared clinical outcome associated with all calibration cell fraction datasets assigned to the TME cell state.

5. The method of claim 2, wherein selecting a treatment for the patient based on the assigned TME cell state comprises:
a. selecting an immune checkpoint blockade treatment if the assigned TME cell state is indicative of an immune-enriched tumors; and
b. selecting a treatment comprising administration of an active compound targeting a molecular pathway specific to an immature malignant cell state if the assigned TME cell state is indicative of a genomically less differentiated tumor.
